(19) United States
(12) Patent Application Publication
(10) Pub. No.: US 2018/0360422 A1
(43) Pub. Date: Dec. 20, 2018

(54) ULTRASOUND IMAGING SYSTEMS HAVING IMPROVED TRANSDUCER ARCHITECTURES

(71) Applicant: General Electric Company, Schenectady, NY (US)

(72) Inventors: Kenneth Wayne Rigby, Clifton Park, NY (US); Ying Fan, Schenectady, NY (US); Naresh Kesavan Rao, Clifton Park, NY (US); Christopher Robert Hazard, Schenectady, NY (US)

(21) Appl. No.: 15/624,482

(22) Filed: Jun. 15, 2017

Related U.S. Application Data
(63) Continuation of application No. 15/624,373, filed on Jun. 15, 2017.

(51) Int. Cl. A61B 8/00; A61B 8/08; A61B 8/14

(52) U.S. Cl. CPC A61B 8/4494; A61B 8/14; A61B 8/5207

(57) ABSTRACT

A transducer assembly is provided. The transducer assembly includes a routing layer. The transducer assembly further includes a plurality of transducer elements arranged on a first side of the interposer. The transducer assembly also includes a first application specific integrated circuit (ASIC) arranged vertically below the plurality of transducer elements and on a second side of the interposer, wherein the first ASIC comprises a plurality of signal conditioning circuits.

### CROSS-REFERENCE TO RELATED APPLICATIONS

The present application is a continuation of U.S. application Ser. No. 15/624,373, entitled “Ultrasound Imaging Systems Having Improved Transducer Architectures,” and filed Jun. 15, 2017, the entirety of which is incorporated by reference herein for all purposes.

### BACKGROUND

The subject matter disclosed herein relates generally to ultrasound imaging, and more specifically to improved transducer assembly architectures, techniques for building such architectures and techniques for efficiently producing ultrasound images.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Medical diagnostic ultrasound is an imaging modality that employs ultrasound waves to probe the acoustic properties of the body of a patient and produce a corresponding image. Generation of the ultrasound wave pulses and detection of the returning echoes is typically accomplished via a plurality of transducer elements located in an ultrasound assembly. The ultrasound assembly, which includes the transducer elements, acoustic matching layers, signal connections and various other components, including in some cases electronic circuitry, is commonly referred to as an ultrasound probe or ultrasound transducer. As used herein, the terms “probe” and “transducer assembly” are used interchangeably. The transducer elements in a probe typically include electromechanical elements capable of converting electrical energy into mechanical energy for transmission and capable of converting mechanical energy into electrical energy for receiving purposes.

Ultrasound imaging systems generally employ an array of transducer elements to transmit the ultrasound beam and subsequently to receive the reflected beam or echo from the object under interrogation (e.g., an organ or region of a patient). As will be appreciated, the array of transducer elements may be arranged in a one- or two-dimensional array. “Two-dimensional” (2D) ultrasound probes typically have rectangular sensing elements in which both dimensions of the element face are of order of the wavelength at the design operating frequency. The elements are usually arranged in a rectangular grid for ease of manufacture, although triangular grids or other geometries have also been used. A 2D probe can be used to scan electronically in two dimensions forming a three-dimensional (“volume”) image. In comparison, the rectangular elements in “one-dimensional” (1D) probes have one dimension which is much larger than a wavelength. These probes are scanned electronically in only one dimension forming a two-dimensional (“slice”) image.

A probe's aperture is the area spanned by its transducer elements. The aperture dimensions are selected based on the intended application for the probe. For example, a cardiac probe is typically small enough to be used between a subject's ribs. Abdominal and breast probes are typically much larger. For a given transducer aperture area, a 2D probe utilizes many more elements than does a 1D probe, of the order of N² versus N elements, where N might be 64 or more. This is a major complication, since connecting each element in a 2D array to beamforming circuitry in a console would employ an impractically bulky cable bundle (or impractically large wireless transmission bandwidth.)

One conventional solution to this limitation is to group the 2D elements into “subapertures,” to partially beamform the element signals within each subaperture to form a subaperture signal, and to bring this smaller number of subaperture signals back to the console, where they are combined to produce one or more beamsums. A fixed pattern of subaperture groupings is the simplest architecture for a transducer array, since it can be implemented by hardwired connections between the transducer elements and the subaperture beamforming circuitry. However, it is often desirable to be able to configure the subaperture groupings programmatically. For instance, an application-specific integrated circuit (ASIC) implementing the subaperture processing is expensive to design and fabricate, so it is desirable to use one ASIC design for more than one 2D probe design each of which could benefit from different arrangements of subapertures. Further, for some types of probes, it can be advantageous to divide the aperture into substantially different sizes of subapertures, rather than employing subapertures of approximately the same size.

FIG. 1 illustrates a simplified block diagram of one potential architecture for an ultrasound system 10 utilizing subaperture processing. Specifically, an M-by-N crossbar switch 12, also known as a “crosspoint switch” or sometimes “matrix switch,” is a circuit with M inputs and N outputs that allows any of the input signals to be routed to any of the output signals. As illustrated, M is the number of transducer elements 14 and N is the number of system channels, with M>>N. The crossbar switch 12 allows more than one input to be connected to the same output. Implicit in this architecture of the system 10 is that the delayed element signals at the outputs of delay blocks 16 can be summed electrically by connecting them to a common wire. As appreciated, the architecture of the system 10 allows any combination of delayed element signals, represented by individual delay blocks 16, to be summed together, producing N signals which are sent to the console 18 either wirelessly or over a probe cable and are further beamformed by the console 18. The elements 14 corresponding to each summed element signal represent a subaperture, typically a contiguous, filled region. However, this architecture has several drawbacks.

As will be appreciated, an M-by-N crossbar 12 may require on the order of M×N switches. A typical 2D abdominal probe might have M=10,000 elements and a typical ultrasound console might have N=200 channels, so that two million switches may be required to implement the crossbar 12. This would require a large area on a silicon device, which may be undesirable as any electronics used in the ultrasound probe increases the bulk of the probe and thus its ease of use. In addition, the cost of a silicon device, and the power consumed by it, increases with its area. The control hardware needed to specify the positions of each switch increases the system cost, complexity, size and power consumption. Further, the delay block 16 must support the maximum possible beamforming delay across the largest subaperture to which it might be attached. That delay structure must be reproduced M times, so it also represents a large area on the silicon implementation.

FIG. 2 illustrates a simplified block diagram of another known ultrasound system 20 utilizing subapertures. A set of Q “subaperture processors 22” is provided, each of which is connected to a fixed subset 24 of P transducer elements 14. Each subaperture processor 22 applies beamforming time delays to the element signals connected to it and outputs a subaperture signal. The Q subaperture signals are routed to N system channels through a Q-by-N crossbar 26. The crossbar 26 is generally needed only for the case Q>N. The crossbar 26 allows the outputs of two or more subaperture processors 22 to be summed (by connecting them to a common wire, as described with regard to FIG. 1). The system 20 generally requires that each subaperture processor 22 support the largest beamforming time delay required across the set of elements in all of the subapertures. This may be undesirable.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the present disclosure. Indeed, the disclosed techniques may encompass a variety of forms that may be similar to or different from the embodiments set forth below.

In one embodiment, a transducer assembly is provided. The transducer assembly includes a routing layer. The transducer assembly further includes a plurality of transducer elements arranged on a first side of the interposer. The transducer assembly also includes a first application specific integrated circuit (ASIC) arranged vertically below the plurality of transducer elements and on a second side of the interposer, wherein the first ASIC comprises a plurality of signal conditioning circuits.

In another embodiment, a transducer assembly is provided. The transducer assembly includes a first application specific integrated circuit (ASIC). The transducer assembly also includes a second application specific integrated circuit (ASIC). The transducer assembly further includes a subaperture comprising a plurality of transducer elements, wherein a first portion of the subaperture is coupled to subaperture processing circuitry on the first ASIC and a second portion of the subaperture is coupled to subaperture processing circuitry on the second ASIC.

In another embodiment, a transducer assembly is provided. The transducer assembly includes a plurality of transducer elements. The transducer assembly further includes a first application specific integrated circuit (ASIC) and a second application specific integrated circuit (ASIC). The transducer assembly also includes a routing layer communicatively coupled to the plurality of transducer elements and each of the first ASIC and the second ASIC.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a simplified block diagram of a conventional ultrasound system utilizing subapertures.

FIG. 2 is a simplified block diagram of another conventional ultrasound system utilizing subapertures.

FIG. 3 is a block diagram an ultrasound system including a transducer array configured in accordance with embodiments of the present invention.

FIGS. 4A and 4B illustrate an example of a summer that may be utilized in the system of FIG. 3, in accordance with embodiments of the present invention.

FIGS. 5A and 5B illustrate an example of a grid of summers and element nodes, in accordance with embodiments of the present invention.

FIG. 6A illustrates an example of a type-1A subaperture, and FIG. 6B illustrates an example of a first-level summer which implements the subaperture processing for a type-1A subaperture, in accordance with embodiments of the present invention.

FIG. 7A illustrates an example of a type-1B subaperture, and FIG. 7B illustrates the first-level summers which implement the subaperture processing for a type-1B subaperture, in accordance with embodiments of the present invention.

FIG. 8A illustrates an example of a type-2 subaperture, and FIG. 8B illustrates an example of a two-level summer architecture which implements the subaperture processing for a type-2 subaperture, in accordance with embodiments of the present invention.

FIGS. 9A and 9B illustrate graphs describing advantages of utilizing a two-level summer architecture, in accordance with embodiments of the present invention.

FIG. 10 shows a block diagram of a switch topology, in accordance with embodiments of the present invention.

FIG. 11 illustrates different topologies of 2-by-2 subapertures, in accordance with embodiments of the present invention.

FIG. 12 illustrates different topologies of 6-by-2 subapertures, in accordance with embodiments of the present invention.

FIG. 13 illustrates different topologies of 3-by-3 subapertures, in accordance with embodiments of the present invention.

FIG. 14 illustrates an unroutable tiling, in accordance with embodiments of the present invention.

FIG. 15 illustrates a routed tiling, in accordance with embodiments of the present invention.

FIG. 16 illustrates a table of possible subaperture dimensions for a tiling, in accordance with embodiments of the present invention.

FIG. 17 illustrates tiling of an aperture, in accordance with embodiments of the present invention.

FIG. 18 illustrates tiling of another aperture, in accordance with embodiments of the present invention.

FIGS. 19A-19F illustrate a central region of subapertures, constructed in accordance with embodiments of the present invention.

FIGS. 20, 21 and 22 illustrate additional components of a translatable aperture, a vertical core including a translatable core, constructed in accordance with embodiments of the present invention.

FIGS. 23, 24, 25 and 26 illustrate additional components of a translatable aperture, including vertical translation of a core and horizontal translation of a vertical core, constructed in accordance with embodiments of the present invention.

FIG. 27 illustrates an aperture divided among multiple subaperture processing ASICs, in accordance with embodiments of the present invention.

FIG. 28 is a block diagram of a design utilizing multiple subaperture processing ASICs, in accordance with embodiments of the present invention.

FIG. 29 illustrates a routing layer used to route signals between multiple subaperture processing ASICs of an aperture, in accordance with embodiments of the present invention.

FIG. 30 is a cross-sectional view of a portion of a transducer assembly, including transducer elements, an interposer layer and subaperture processing circuitry, illustrating an interposer layer used to couple signals between multiple subaperture processing ASICs, in accordance with embodiments of the present invention.

FIG. 31 is a cross-sectional view of a portion of a transducer assembly, illustrating an interposer layer used to couple transducer elements arranged at one pitch to subaperture processing circuitry arranged at a second pitch, in accordance with embodiments of the present invention.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time-consuming but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

Technical effects of the invention include unique hardware architectures for ultrasound transducer assemblies and their related components. More particularly, various arrangements of switches and summers which provide flexibility in the choice of subaperture groupings without using crossbar switches are disclosed. As used herein, a “crossbar” refers to a switch having M inputs and N outputs (M-by-N), where M is greater than 1. By using two levels of summation in the transducer array, the disclosed architectures minimize the amount of beamforming time-delay required by a summer, which reduces the system cost, complexity, area and power consumption.

Further, by employing a routing layer (e.g., a flexible circuit or an interposer) between the elements of the transducer array and the circuits that may be fabricated on different and discrete ASICs (application-specific integrated circuits), the pitch of transducer elements is decoupled from the pitch of the circuit elements. This avoids the cost of designing and manufacturing different ASICs for different transducer acoustic designs. In certain embodiments, the routing layer also allows for some transducer elements to be switchably connected to circuit elements on two or more different ASICs, simplifying the control of subapertures that extend past ASIC boundaries.

Still further, a methodology for generating suitable, useful arrangements of subapertures is disclosed. While subdividing a rectangular aperture into a desired number of nearly equal-sized rectangular subapertures is fairly straightforward, subdividing a rectangle into a desired number of non-uniform rectangular subapertures is typically non-trivial and thus, may lead to a considerable amount of time and effort in each unique design. In accordance with embodiments described herein, methods for generating a class of subaperture arrangements algorithmically by setting certain rules and variables for consideration of the subaperture arrangement are provided. Generating a set of subapertures algorithmically is advantageous because it reduces the time and expense that may be required to find suitable arrangements of subapertures. It may also reduce the likelihood of errors in the subaperture design. Still further, the system control that may be required to specify an arrangement of subapertures can be simplified, reducing the cost and power consumed by the control circuitry.

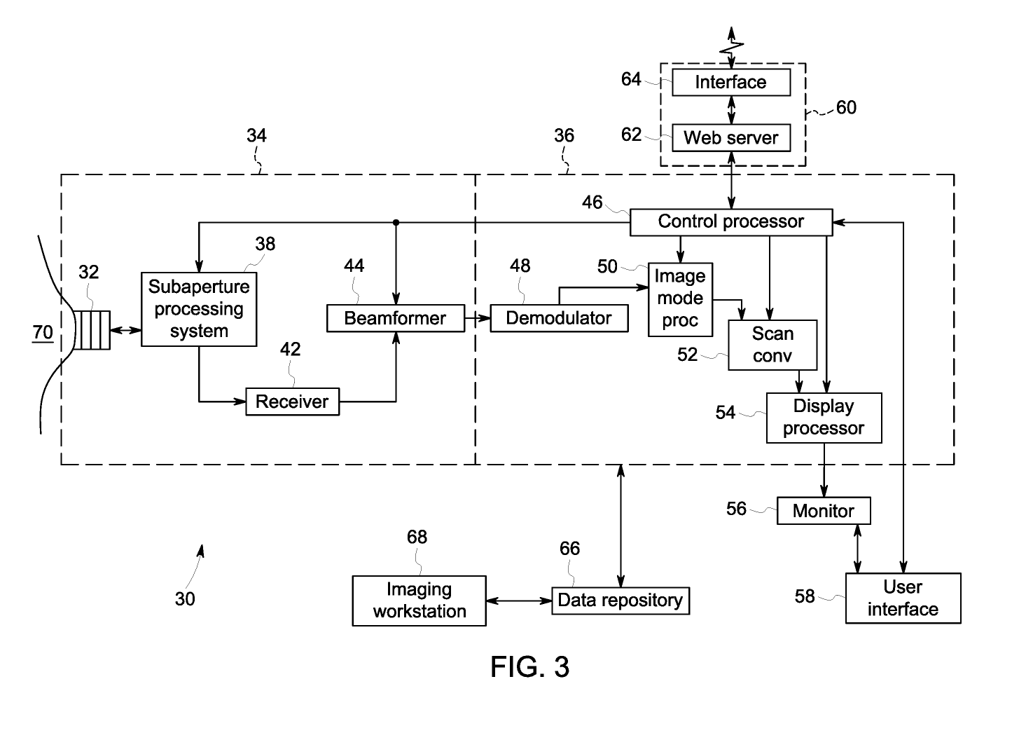

Turning again to the figures, FIG. 3 is a more detailed block diagram of one example of an ultrasound system 30 including a transducer array 32 configured in accordance with one embodiment of the present invention. As illustrated, the ultrasound system 30 may include an acquisition subsystem 34 and a processing subsystem 36. The acquisition subsystem 34 may include the transducer array 32 (having a plurality of transducer array elements and associated transmitter control circuitry), a subaperture processing system 38, a receiver 42, and a beamformer 44. The processing subsystem 36 may include a control processor 46, a demodulator 48, an imaging mode processor 50, a scan converter 52 and a display processor 54. The display processor 54 may be further coupled to a display monitor 56 for displaying images, while the user interface 58 may interact with the control processor 46 and the display monitor 56. The control processor 46 may also be coupled to a remote connectivity subsystem 60 to provide remote access to at least a portion of ultrasound system 30. The remote connectivity subsystem 60 may include e.g., a web server 62 and a remote connectivity interface 64. The processing subsystem 36 may be further coupled to a data repository 66 configured to receive and store ultrasound image data. The data repository 66 interacts with an imaging workstation 68.

The aforementioned components may include dedicated hardware elements such as circuit boards with digital signal processors or may represent functional software components designed for execution on a general or special-purpose computer or processor. The various components may be combined or separated according to various embodiments of the invention. Thus, it should be appreciated that the present ultrasound system 30 is provided by way of example, and the present techniques are in no way limited by the specific system configuration.

In the acquisition subsystem 34, the transducer array 32 may be contained within a transducer assembly that is intended to be placed in contact with a patient or subject 70. The transducer array 32 may be coupled to the subaperture processing system 38. The subaperture processing system 38 may operate under control of the control processor 46 in the processing subsystem and provides acquired data to the input of the receiver 42. The output of the receiver 42 is configured as an input to the beamformer 44. As illustrated, the beamformer 44 further may be coupled to the input of the demodulator 48. The beamformer 44 also may be coupled to the control processor 46, as shown in FIG. 3.

In the processing subsystem 36 which may be contained within a local or remote console, the output of demodulator 48 is coupled to an input of an imaging mode processor 50. In addition to providing control signals to configure the subaperture processing system 38, the control processor 46 interfaces with the imaging mode processor 50, the scan converter 52, the display processor 54 and the transmitter control circuitry in the probe assembly (not shown). An output of imaging mode processor 50 is coupled to an input of scan converter 52. An output of the scan converter 52 is coupled to an input of the display processor 54. The output of display processor 54 is coupled to the monitor 56.

During operation, the ultrasound system 30 transmits ultrasound energy into the subject 70 and receives and processes backscattered ultrasound signals from the subject 70 to create and display an image. To generate and transmit a beam of ultrasound energy, the control processor 46 sends command data to the transmitter circuitry in the transducer assembly to create a beam of a desired shape originating from a certain point at the surface of the transducer array 32 at a desired steering angle. The transmit signals are set at certain levels and phases with respect to each other and are provided to individual transducer elements of the transducer array 32. The transmit signals excite the transducer elements to emit ultrasound waves with the corresponding phase and level relationships. As a result, a transmitted beam of ultrasound energy is formed in a subject 70 within a scan plane along a scan line when the transducer array 32 is acoustically coupled to the subject 70 by using, for example, ultrasound gel.

The transducer array 32 is a two-way transducer. When ultrasound waves are transmitted into a subject 70, the ultrasound waves are backscattered off the tissue and blood samples within the subject 70. The transducer array 32 receives the backscattered waves at different times, depending on the distance into the tissue they return from and the angle with respect to the surface of the transducer array 32 at which they return. The transducer elements convert the ultrasound energy from the backscattered waves into electrical signals.

The electrical signals are then routed to the subaperture processing system 38, which combines the element signals into subaperture signals and routes them to the receiver 42. The receiver 42 amplifies and digitizes the signals and provides other functions such as gain compensation.

The digitized subaperture signals are sent to the beamformer 44. The control processor 46 sends command data to the beamformer 44. The beamformer 44 uses the command data to form a receive beam originating from a point on the surface of the transducer array 32 at a steering angle typically corresponding to the point and steering angle of the previous ultrasound beam transmitted along a scan line. The beamformer 44 operates on the appropriate subaperture signals by performing time-delaying and summation, according to the instructions of the command data from the control processor 46, to create received beam signals corresponding to sample volumes along a scan line in the scan plane within the subject 70.

The received beam signals are sent to the processing subsystem 36. The demodulator 48 demodulates the received beam signals to create pairs of I and Q demodulated data values corresponding to sample volumes within the scan plane. Demodulation is accomplished by comparing the phase and amplitude of the received beam signals to a reference frequency. The I and Q demodulated data values preserve the phase and amplitude information of the received signals.

The demodulated data is transferred to the imaging mode processor 50. The imaging mode processor 50 generates imaging parameter values from the demodulated data in scan sequence format. The imaging parameters may include parameters corresponding to various possible imaging modes such as B-mode, color velocity mode, spectral Doppler mode, and tissue velocity imaging mode, for example. The imaging parameter values are passed to the scan converter 52. The scan converter 52 processes the parameter data by performing a translation from scan sequence format to display format. The translation includes performing interpolation operations on the parameter data to create display pixel data in the display format.

The scan converted pixel data is sent to the display processor 54 to perform any final spatial or temporal filtering of the scan converted pixel data, to apply grayscale or color to the scan converted pixel data, and to convert the digital pixel data for display on the monitor 56. The user interface 58 is coupled to the control processor 46 to allow a user to interface with the ultrasound system 30 based on the data displayed on the monitor 56.

### Hardware Architecture

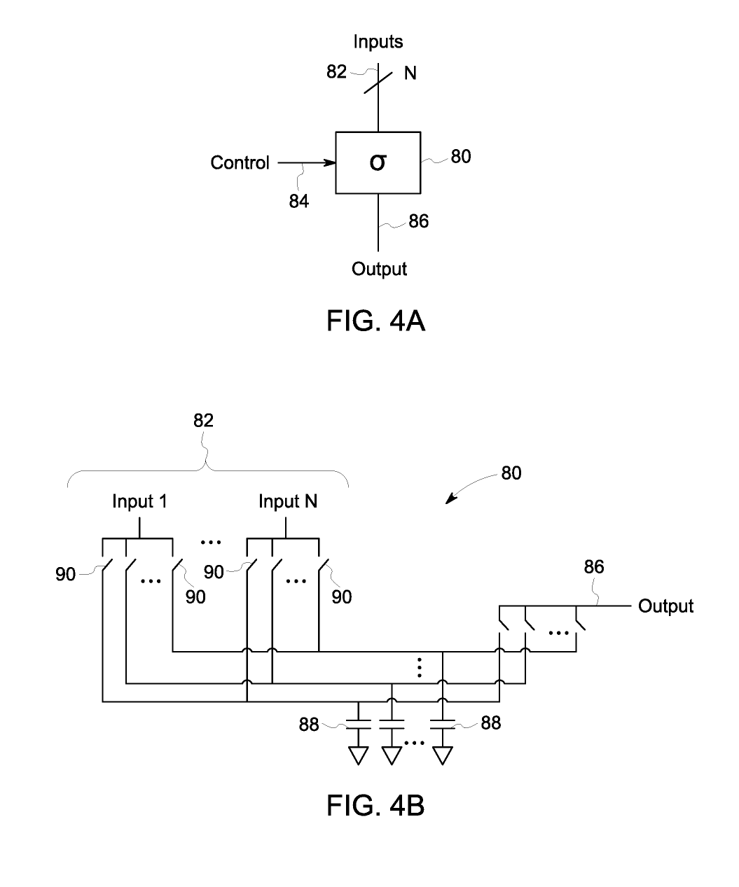

In discussing various embodiments of the present invention, certain features will be described below, in order to provide the building blocks on which the embodiments described herein are based. FIG. 4A shows a delay-and-sum block, which we will be referred to herein as a “summer” 80, with N analog inputs 82, control lines 84, and a single analog output 86. FIG. 4B shows schematically one implementation of such a summer 80 as a set of capacitors 88 arranged as a capacitor delay line with switches 90 connecting the capacitors 88 to the inputs 82 and output 86. As with the subaperture processors 22 discussed with regard to FIG. 2, the function of the summer 80 is to delay-and-sum a group of signals (e.g., inputs 82), forming a subaperture signal (e.g., output 86). By briefly closing each of the switches 90 connected to an input 82 at various times, the voltage on the input 82 is used to charge the capacitors 88, with the stored charge proportional to the voltage, Qᵢ=C Vᵢ. For instance, the switches 90 coupled to the first input 82 (e.g., Input 1), may be successively closed at times t₁, t2, etc., such that the voltage present on Input 1 at those times can be stored as charges on the respective capacitor 88 routed to each switch 90. The delay-and-sum operation is implemented by closing the input switches 90 in such a way that a given capacitor 88 is connected to each input 82 at the appropriate time so that it accumulates charges proportional to the input voltage at the desired time-delay for each input 82.

The number of capacitors 88 is proportional to the maximum desired time-delay across the inputs 82, since the charges resulting from sampling, e.g., at Input 1, are stored on separate capacitors 88 until all of the inputs 82 which contribute to the first sample from Input 1 have arrived and have been accumulated into the corresponding capacitor 88. Once this occurs, the switch 90 connecting this capacitor 88 to the output 86 can be closed, draining the summed charge to the output line and freeing that capacitor 88 for reuse. One important feature of this structure is that the outputs 86 from two or more summers 80 can be added, with minor additional support circuitry, by connecting those outputs 80 to a common wire, as discussed further below. Draining the charge stored in the output capacitor 88 of each summer 80 to a common wire produces a net charge proportional to the sum of the charge on each summer 80, i.e., to the sum of the calculated delayed-and-summed output voltage for each summer 80.

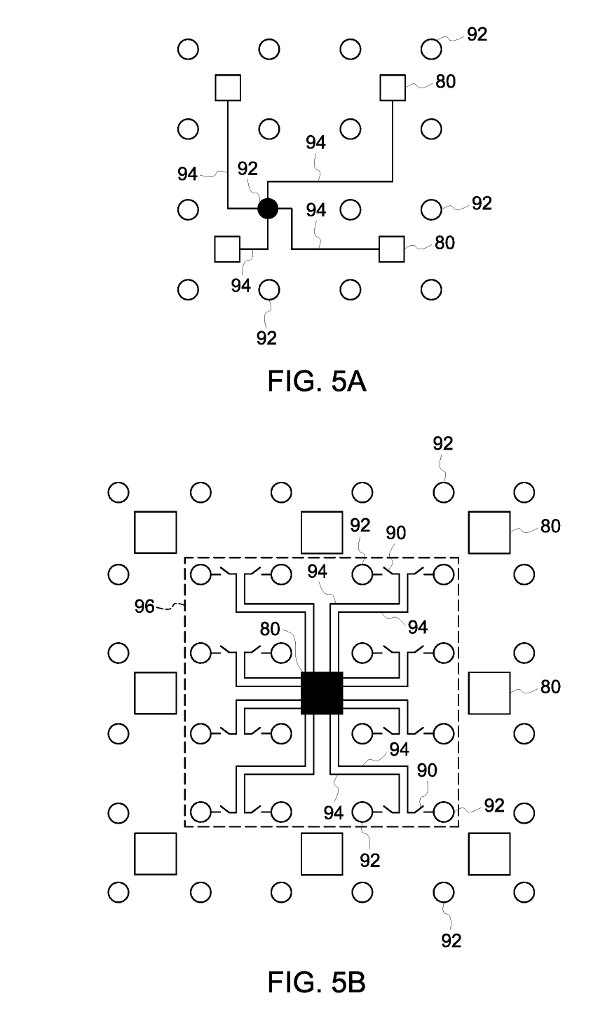

FIG. 5A shows a small portion of a square grid of transducer element nodes 92 (depicted as circles) and a square grid of summers 80 (depicted as squares or rectangles). The element node 92 represents the end of the electrical signal path including a transducer element and any analog receive signal conditioning circuitry, such as preamplifiers, time-gain-compensation amplifiers, transmit-receive switches, voltage-to-charge amplifiers, etc. As will be described further below, the transducer elements may be separated from the signal conditioning circuitry and the summers. That is, the signal conditioning circuitry and summers may be located on one or more ASICs that are discrete and physically independent from the transducer array comprising the transducer elements of a particular element node 92. The transducer elements of a particular element node 92 may be electrically coupled to the associated summers 80 and signal conditioning circuitry through a flexible circuit or interposer layer, as described in greater detail below with regard to 28-31.

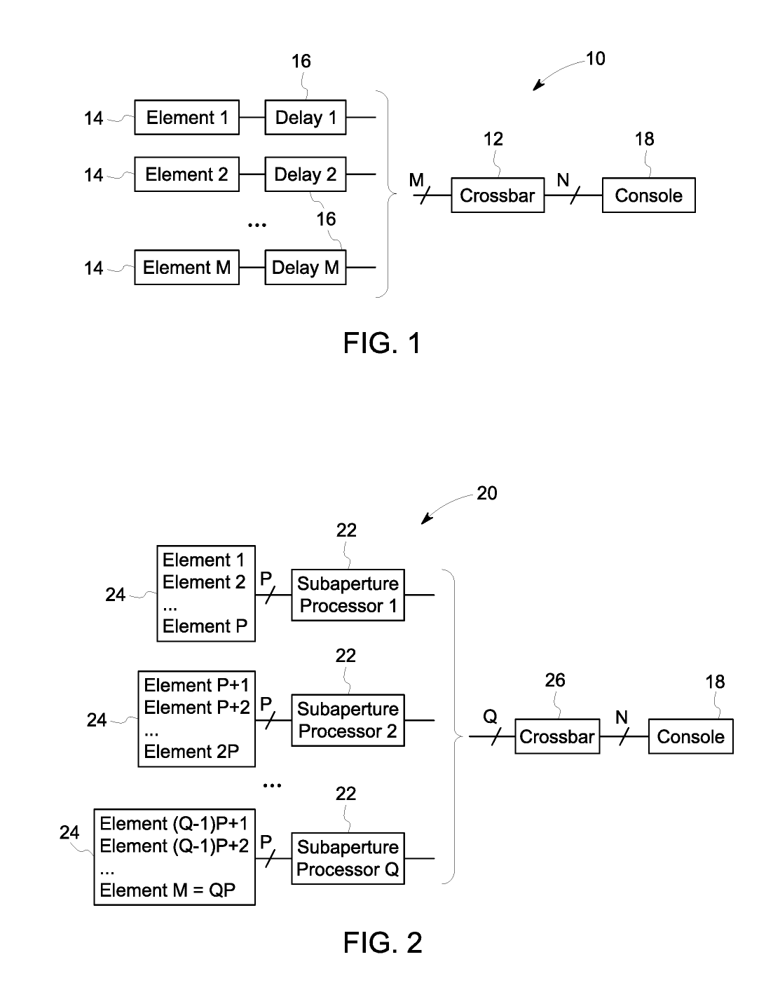

One important feature of the arrangement shown in FIG. 5A is that there are only one-fourth as many summers 80 as element nodes 92 in contrast to the subaperture processing architecture illustrated in FIG. 1. Each element node 92 can be electrically connected to any of the four surrounding summers 80 through the input switches 90 (not shown) of the summers 80. FIG. 5A shows the possible summer connections 94 for one element node 92 (depicted as a filled circle). This connectivity of element nodes 92 and summers 80 requires that each summer 80 connect, through its input switches 90, to the sixteen nearest surrounding element nodes 92, as indicated by the dashed square 96 in FIG. 5B. For clarity, the switches 90 are drawn in FIG. 5B outside the rectangle representing the summer 80, although functionally they are part of the summer 80. The summer outputs 86 are not shown, nor are the paths for the summer control lines 84. FIG. 5B shows the connections 94 for only one summer 80 (depicted as a filled rectangle), but this pattern is repeated for every summer 80 in the grid of summers 80, except for element nodes 92 and summers 80 at the boundaries of the circuitry, which have fewer connections.

While FIG. 5A and FIG. 5B show the element nodes 92 and summers 80 on a square grid, embodiments provided herein do not require that the transducer elements connected to element nodes 92 lie on a square grid. Further, the spacing of the transducer elements connected to element nodes 92 may not be equal to that of the spacing of the grid of element nodes 92. Still further, the transducer elements connected to element nodes 92 need not be arranged in a square. The signals from the grid of transducer elements can be routed over a flexible printed circuit or interposer layer to the inputs of an ASIC implementing the subaperture processing, which allows the spacing of the electrical traces at either end to be different, as will be described in further detail below. If the ASIC is intended to lie directly behind the transducer elements, an interposer layer (described below) can make electrical connections between the electronic structures on a grid of one size and the transducer elements on a grid of a different size. The surface area of the ASIC can be smaller or larger than the surface area of the transducer aperture. The ASIC can be connected to transducer elements with different apertures or different element spacing by using different interposer structures. This avoids the cost of designing and manufacturing different ASICs for different transducer array designs. Thus, only the topology of connections 94 between the element nodes 92 and summers 80 is notable and the summers 80 need not physically lie within the grid of element nodes 92, providing greater design flexibility.

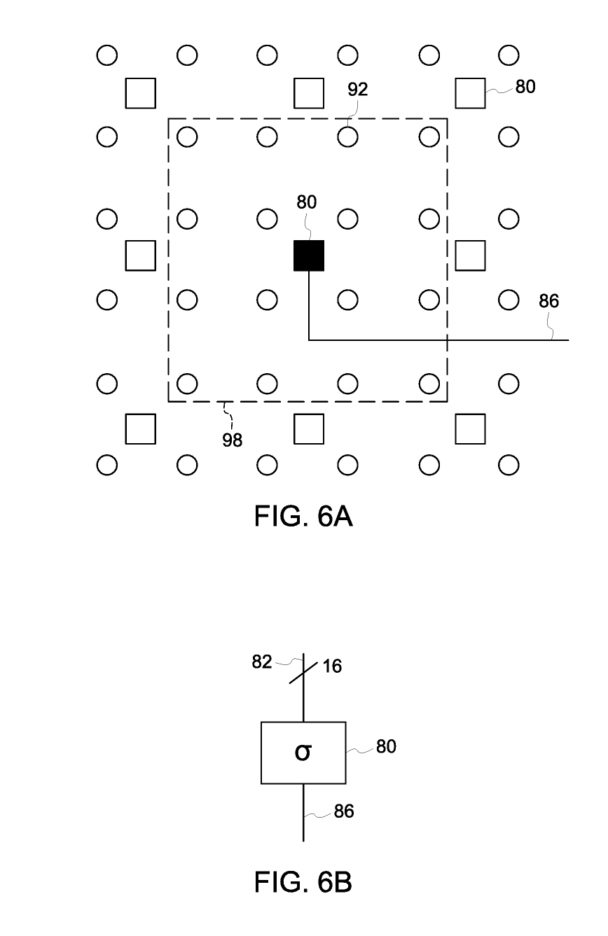

The filled square in FIG. 6A represents a single summer 80, and the line connected to it represents its output signal path 86. The dashed line 96 shows the sixteen element nodes 92 to which this summer 80 can connect. FIG. 6B shows the corresponding block diagram representation, wherein the control signals are not shown. The summer 80 is designed to support the maximum beamforming time delay across the sixteen element nodes 92. The beamforming time delay depends on the dimensions of the transducer elements connected to the element nodes 92, not the spacing of the nodes 92 in the circuit implementation. The maximum time delay is roughly proportional to the largest diagonal of the rectangular area spanned by the transducer elements connected to the nodes 92 which are to be delayed and summed. A single summer 80 can delay-and-sum the signals for any number of element nodes 92 between one and sixteen, or it can be configured so that it does not connect to any element nodes 92. Typically, it will delay-and-sum signals for transducer elements connected to nodes 92 which form a filled rectangular group, though that is not required.

As used herein, the set of element nodes 92 which are summed through the same summer 80, may be referred to as a “subaperture,” such as the subaperture 98, depicted by the dashed lines of FIG. 6A. The subaperture 98 is referred to as a “4-by-4” subaperture, since it consists of a rectangular region four element nodes 92 horizontally and four element nodes 92 vertically. A single summer 80 can be configured to create a variety of subapertures, such as a “1-by-4,” “2-by-2,” “4-by-4,” etc.

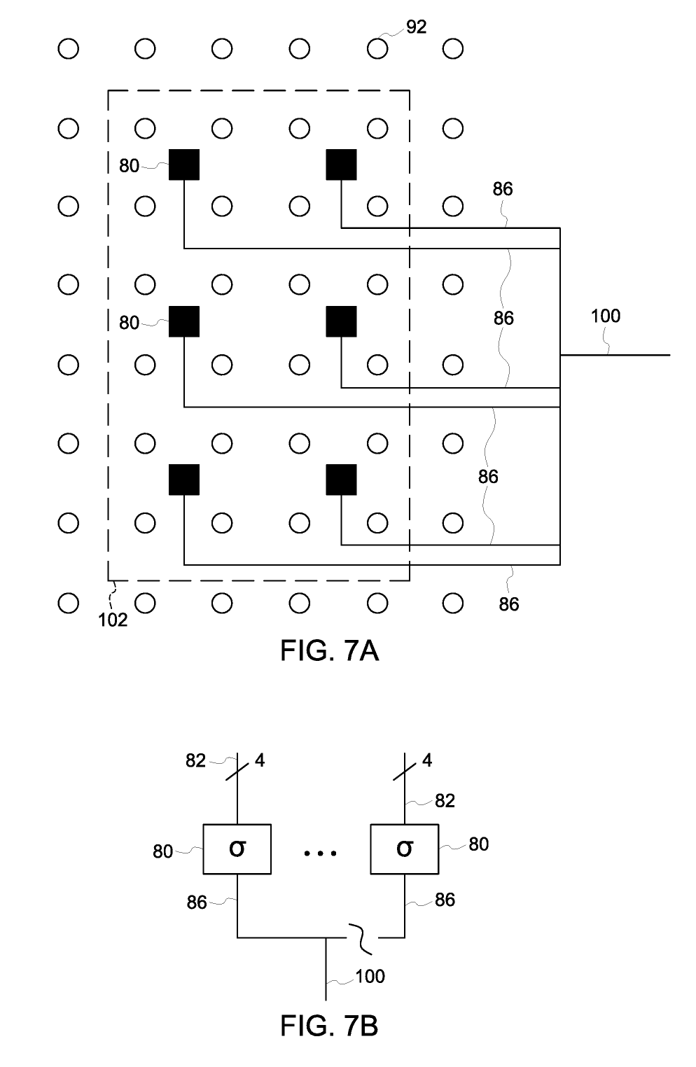

The outputs 86 of two or more summers 80 can be summed to form subapertures with one or both dimensions larger than four element nodes 92, as shown in FIG. 7A. In this example, the outputs 86 of six summers 80 each forming a 2-by-2 subaperture are summed by connecting the outputs 86 of the respective summers 80 to a single wire 100, as previously described. The result is a 4-by-6 subaperture 102, as indicated by the dashed line. The corresponding block diagram is illustrated in FIG. 7B. As will be understood, each summer 80 supports a delay equal to the maximum required beamforming delay across the set of transducer elements connected to the element nodes 92 enclosed by the dashed line depicting the subaperture 102, since the outputs 86 of the summers 80 are summed without any additional delay. The summers 80 in FIG. 7B are labeled with four inputs 82 to clarify that only four of their inputs 82 are used for the configuration illustrated in FIG. 7A. However, as will be appreciated, each summer 80 is identical to that shown in FIG. 6B, with sixteen inputs 82.

As will be appreciated, arbitrarily large subapertures could be formed using the circuit elements shown in FIG. 6B and FIG. 7B, but disadvantageously, this architecture requires that every summer 80 must support the corresponding large beamforming delay across the subaperture. Instead, in accordance with embodiments of the invention disclosed herein, larger subapertures may be formed by adding a second level of summer structures. By utilizing a two-level summation, the disclosed architectures minimize the amount of beamforming time-delay required by a summer, which reduces the system cost, complexity, area and power consumption.

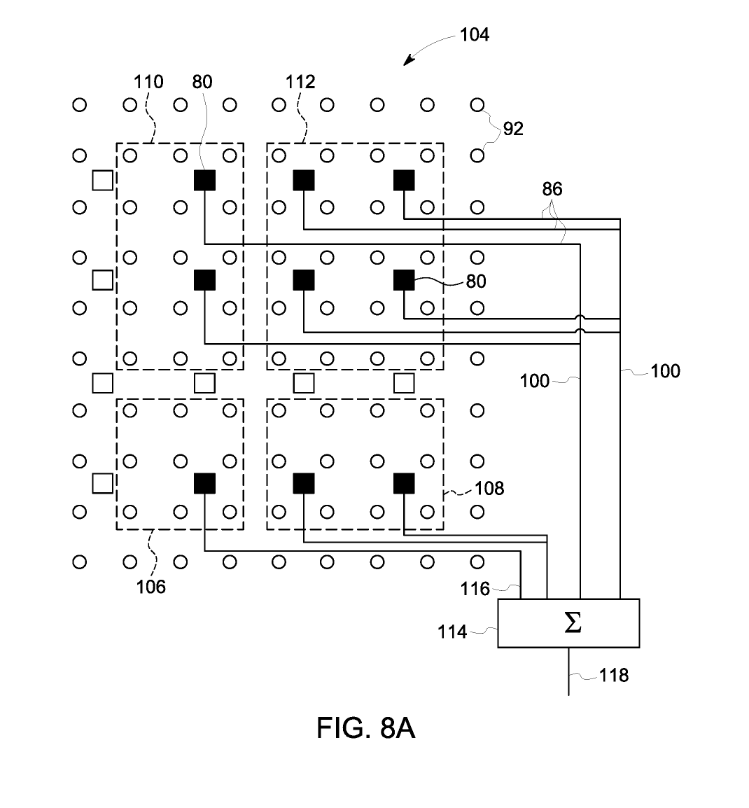

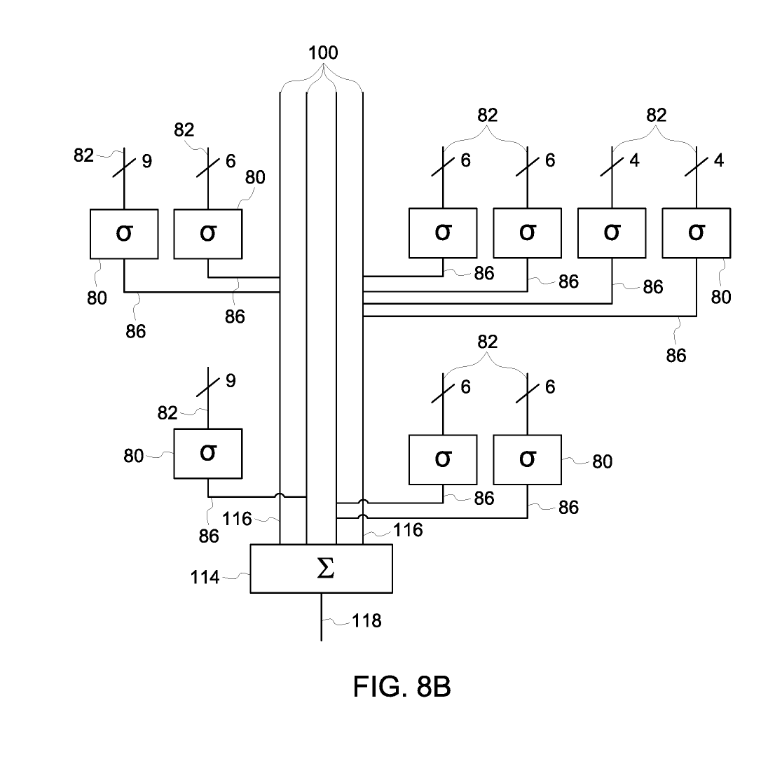

FIG. 8A shows a 7-by-8 subaperture 104, formed by summing—with delay—the outputs 86 of a 3-by-3 subaperture 106, a 4-by-3 subaperture 108, a 3-by-5 subaperture 110 and a 4-by-5 subaperture 112. The filled rectangles indicate the first-level summers 80 that are used to form the internal subapertures 106, 108, 110 and 112. The division of the subaperture 104 into internal subapertures 106, 108, 110 and 112 could, for example, be chosen to minimize the maximum delay required by any first-level summer 80. FIG. 8B shows the corresponding block diagram for this example. The second-level summer 114, which is labeled by Σ to distinguish it from the first-level summers 80, which are labeled by σ, has the same general structure as the first-level summer 80 but will typically have fewer inputs 116 (four are shown, in FIG. 8A and FIG. 8B), and it may support a different maximum delay. Each first-level summer 80 has sixteen inputs, as previously described, although in FIG. 8B the number of inputs 82 to each respective first-level summer 80 shows the number of inputs 82 used for the example configuration shown in FIG. 8A. Each of the outputs 86 from each first-level summer 80 in a respective subaperture (e.g., the 3-by-3 subaperture 106, the 4-by-3 subaperture 108, the 3-by-5 subaperture 110 and the 4-by-5 subaperture 112) may be connected together through a single wire 100, as previously discussed. These wires 100 represent the outputs of the subapertures 106, 108, 110 and 112 that are then used as inputs 116 to the second-level summer 114. By summing and delaying the inputs 116 of the second-level summer 114, a single output 118 is produced.

As used herein, the subapertures formed using one or more summers 80, but only a single level of summation, are referred to as “type 1” subapertures. Type-1 subapertures can be divided into those that use one summer 80, which will be referred to herein as “type 1A” subapertures, and those that use two or more summers 80, which will be referred to herein as “type 1B” subapertures. The block diagram shown in FIG. 6B represents the subaperture processing for the type-1A subaperture illustrated in FIG. 6A. The block diagram shown in FIG. 7B represents the subaperture processing for the type-1B subaperture illustrated in FIG. 7A. A subaperture using two levels of summation is called a “type 2” subaperture. The block diagram shown in FIG. 8B represents the subaperture processing for the type-2 subaperture illustrated in FIG. 8A.

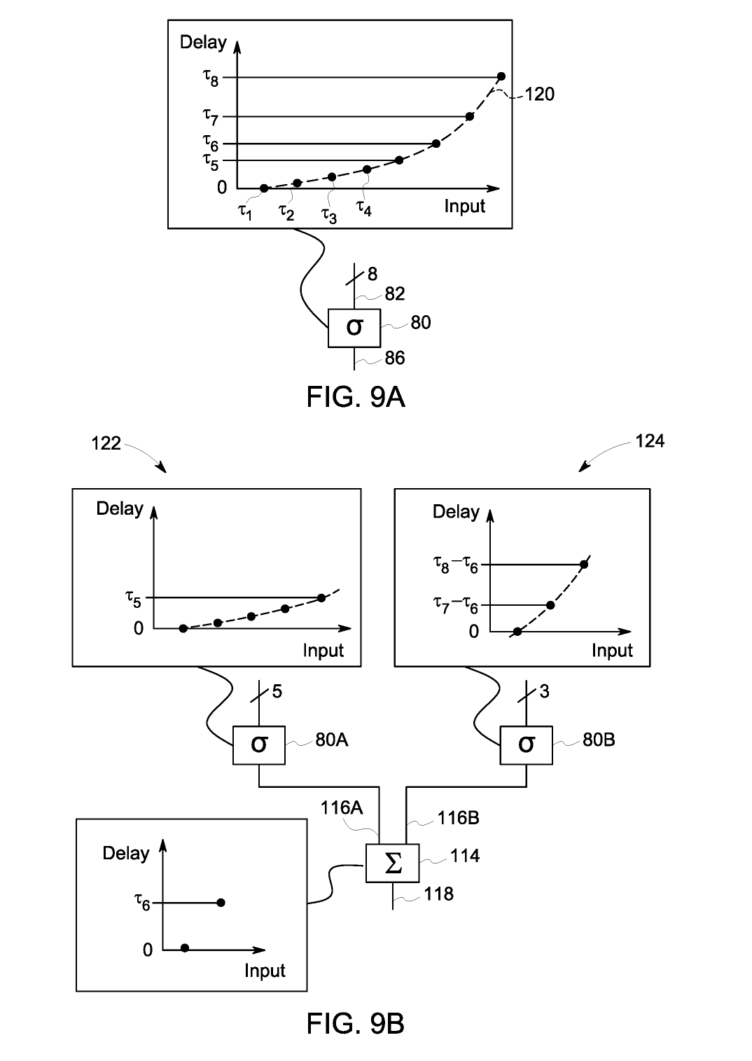

FIGS. 9A and 9B illustrate one advantage of using two levels of summers 80 and 114, as described with reference to FIGS. 8A and 8B. FIG. 9A illustrates a set of eight beamforming time delays (τ₁ through τ₈) in order of increasing magnitude, generally illustrated by line 120. If these delays are implemented by a single summer 80 with eight inputs 82, as shown in the block diagram of FIG. 9A, the summer 80 must support a maximum delay of τ₈, the value of the largest delay minus the smallest delay, which is zero in this example. However, in FIG. 9B, the same set of eight delays (τ₁ through τ₈) are implemented using two levels of summers (80 and 114). The eight delays are divided into two groups 122 and 124. The maximum delay used in the leftmost first-level summer 80A is τ₅, and the maximum delay used in the rightmost first-level summer 80B is τ₈−τ₆. In the second-level summer 114, the left input 116A receives no additional delay and the right input 116B receives an additional delay of τ₆. The net delays produced by the two levels of summers 80 and 114 in FIG. 9B are identical to that produced by the one summer 80 used in FIG. 9A.

The maximum delay used by all three summers 80A, 80B and 114 in FIG. 9B is the larger of τ₆ and τ₈−τ₆, both of which are smaller than τ₈, the maximum delay required using the single summer 80 in FIG. 9A. This example illustrates that implementing the desired delay using two levels of summers 80 and 114 reduces the maximum delay a summer 80 or 114 must support. This tends to reduce the circuit size and control complexity. More summers 80 and 114 are employed, however, so this must also be considered when optimizing the circuitry.

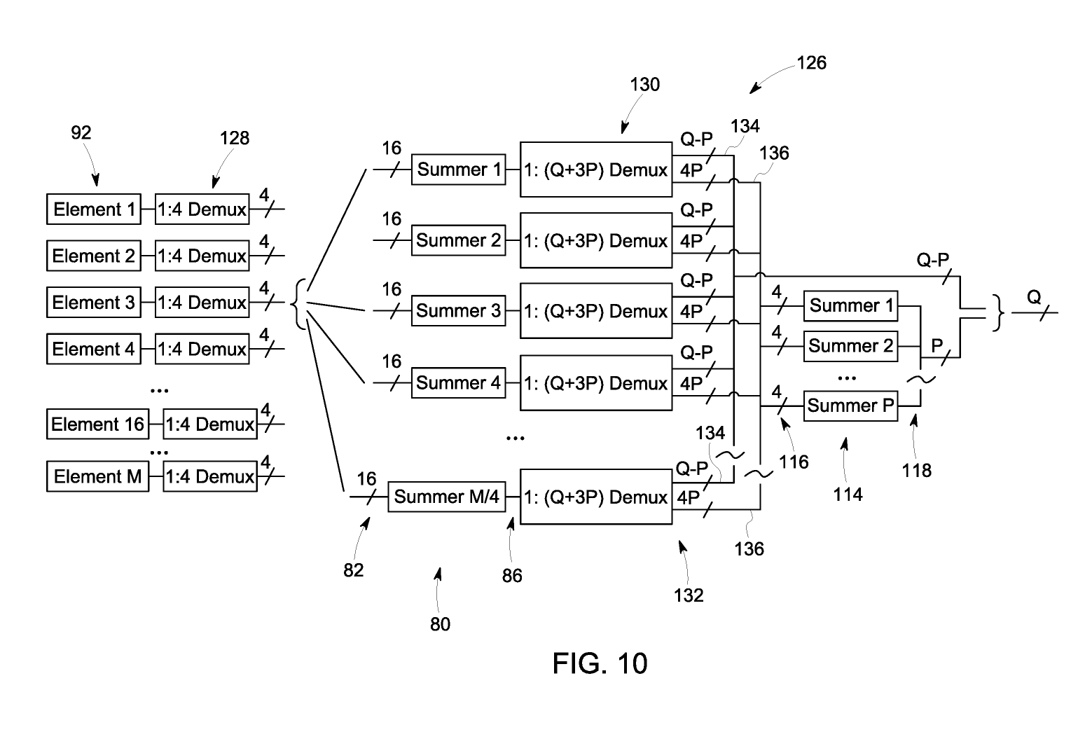

FIG. 10 shows a block diagram of the switch topology for the proposed architecture. The circuitry 126 has inputs for M element nodes 92 and produces Q output signals, i.e., Q subaperture signals. Each element node 92 (except for elements along the transducer array boundaries for implementations that use a single ASIC, or along the boundary of transducer elements connected to an ASIC for implementations that use multiple ASICs) connects through a 1-by-4 demultiplexer 128 to four first-level summers 80, as described previously and illustrated in FIG. 5A. As used herein, a “demultiplexer” refers to a switch having one input and N outputs (1-by-N). There are M/4 first-level summers 80, one for every four element nodes 92. The control for the demultiplexers 128 (not shown) specifies to which of the four first-level summer 80, if any, a given element signal is connected. As described earlier and illustrated in FIG. 5B, each first-level summer 80 has sixteen inputs 82 which connect to sixteen of these demultiplexers 128.

The output 86 of each first-level summer 80 connects to a demultiplexer 130 with (Q+3P) outputs 132. Control for the demultiplexer 130 (not shown) either connects the first-level summer output 86 to one of the demultiplexer outputs 132 or leaves the summer output 86 unconnected. The (Q+3P) outputs 132 for these demultiplexers 130 connect to one of two signal buses 134 or 136: either 1) a bus 134 of (Q−P) signals, which form (Q−P) outputs of the circuit 126, or 2) a bus 136 of 4P signals, which connects to the four inputs 116 of P second-level summers 114. The P outputs 118 of these P second-level summers 114 form the remaining outputs of circuitry 126.

To form a type-1A subaperture, such as the one illustrated in FIG. 6A, the demultiplexers 128 connected to the subaperture's element nodes 92 are configured to connect all the element nodes 92 to one first-level summer 80 calculated or specified in a table. The demultiplexer 130 at that first-level summer's output 86 is configured to route the first-level summer's output 86 to one of the signals in the bus 134 of (Q−P) signals and, thus, to one of the circuitry 126's outputs. This implements the block diagram shown in FIG. 6B.

To form a type-1B subaperture, such as the one illustrated in FIG. 7A, the demultiplexers 128 connected to the subaperture's element nodes 92 are configured to connect each of the element nodes 92 to a group of first-level summers 80 calculated or specified in a table. Each of the corresponding demultiplexers 130 at the first-level summers outputs 86 are configured to route the first-level summer's outputs 86 to the same signal in the bus 134 of (Q−P) signals and, thus, to one of the circuit's outputs. This implements the block diagram shown in FIG. 7B.

To form a type-2 subaperture, such as the one illustrated in FIG. 8A, the sizes of the constituent subapertures are calculated or looked up in a table. For each constituent subaperture, the demultiplexers 128 connected to the constituent subaperture's element nodes 92 are configured to connect each element node 92 to one of the first-level summers 80 calculated or specified in a table, exactly as is done when forming type-1A or type-1B subapertures. The output of one constituent subaperture is formed by connecting the outputs 86 of the corresponding first-level summer 80 or summers 80 to a single line in the bus 136 of 4P signals; that line is connected to an input 116 to one of the second-level summers 114 (e.g., second-level “summer 1” in FIG. 10). The output of a second constituent subaperture is formed similarly, by connecting the outputs 86 of the corresponding first-level summer 80 or summers 80 to a second line in the bus 136 of 4P signals, a line connected to a second input of the same second-level summer 114 (e.g., second-level “summer 1”). These connections are repeated for every constituent subaperture. The second-level summer 114 (e.g., summer 1) delays and sums its inputs, and its output becomes one of the circuitry 126 outputs. This implements the block diagram shown in FIG. 8B.

The circuitry 126 can be configured to form as many as Q subapertures, of which at most P can be type-2 subapertures. As will be appreciated, the bus of P signals can also be used to form type-1 subapertures, if desired, by configuring the second-level summer 114 to apply no time-delays to its inputs 116. The variables, factors and techniques for choosing Q and P are described further below, in accordance with the disclosed embodiments. By way of illustration, the second-level summers 114 in circuit 126 are shown with four inputs, but it will be appreciated that second-level summers with a different number of inputs could be used depending upon such factors as the desired largest size of a subaperture. Similarly, each element node 92 is shown switchably connected to four first-level summers 80 through demultiplexers 128, but a demultiplexer with a different number of outputs could be used depending upon such factors as a trade-off between demultiplexer size and ASIC signal routing complexity.

The number of switches 90 (not illustrated) utilized to implement the demultiplexers 128 and 130 in FIG. 10 is approximately

4M+(M/4)(Q+3P)=(MQ/4)(1+3P/Q+16/Q).

In comparison, implementing the functionality of circuitry 126 using an M-by-Q crossbar, as utilized in the previous systems described with reference to FIGS. 1 and 2, would require approximately MQ switches. As an example, let Q=64 and P=20. The architecture shown in FIG. 10 advantageously utilizes, in this case, only about half as many switches as an implementation using an M-by-Q crossbar.

### Subaperture Orientation

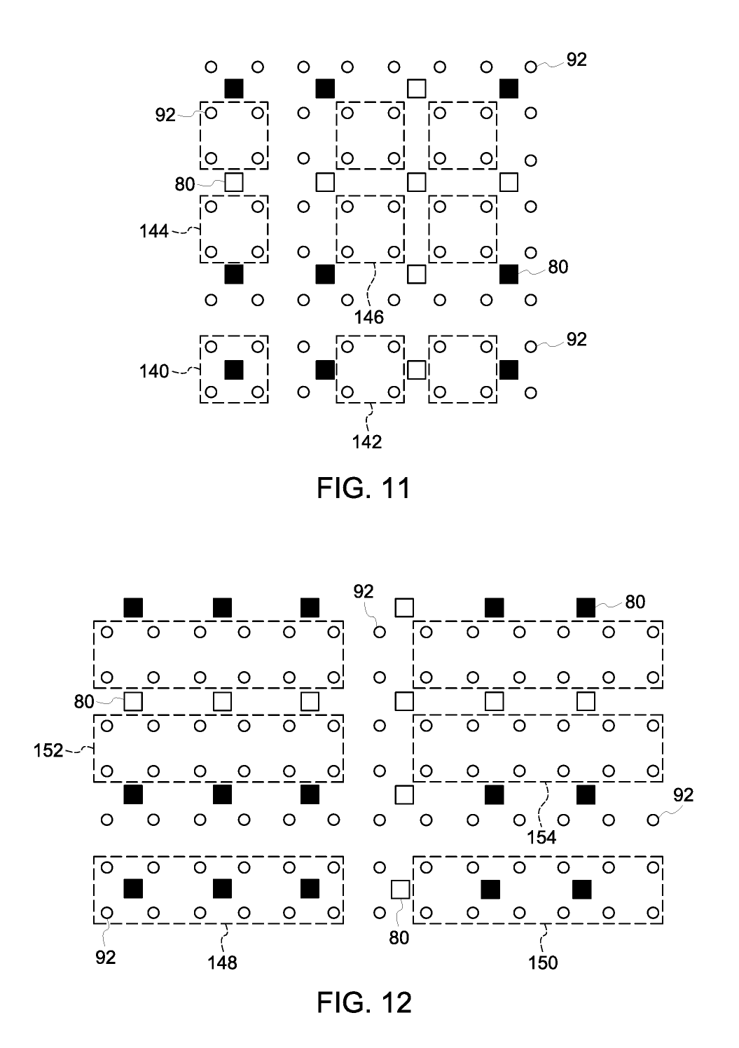

There can be multiple ways to connect the element nodes 92 in a subaperture to a first-level summer 80 or summers 80. For example, the single element node 92 in a 1-by-1 subaperture can connect to any of the four nearest summers 80, as shown in FIG. 5A. The “orientation” of a subaperture is the specification of the summer 80 or summers 80 connected to the element nodes 92 of that subaperture. In figures FIG. 6A, FIG. 7A and FIG. 8A, the summers 80 connected to the element nodes 92 lie inside the dashed lines which mark the element nodes 92 in the subaperture, but this is not required. As illustrated in FIG. 11 and FIG. 12, the summers 80 may lie outside the dashed lines which mark the element nodes 92 in the subaperture. As used herein, the former is referred to as an “internal” summer 80 and the latter is referred to as an “external” summer 80.

This terminology should not be interpreted to mean that the summer circuitry must lie physically within the grid of element nodes, as discussed above. The topology of the connections between summers and elements for the subapertures is independent of where the circuitry lies physically. The summer circuitry need not even lie in the probe assembly.

There are four topologically distinct positions of a subaperture with respect to the grid of summers 80, two positions generated by horizontal displacements and two positions generated by vertical displacements. FIG. 11 illustrates this for a 2-by-2 subaperture; the four distinct positions are drawn as dashed squares labeled 140, 142, 144 and 146. At position 140, only one orientation is possible, that in which the subaperture uses an internal summer 80, illustrated as a filled squared in FIG. 11. At position 142, there are two orientations, one which connects the subaperture's element nodes 92 to the summer 80 on the left of the subaperture, and one which connects the subaperture's element nodes 92 to the summer 80 on right. At position 144, there are two orientations, one using the summer 80 above the subaperture and one using the summer 80 below it. At position 146, there are four orientations, one for each summer 80 at the four corners of the subaperture. Thus there are nine orientations for a 2-by-2 subaperture, each indicated by dashed lines.

As another example, FIG. 12 shows the six orientations of a 6-by-2 subaperture. The four topologically distinct positions of the subaperture with respect to the grid of summers 80 are indicated by the dashed rectangles labeled 148, 150, 152 and 154. Subaperture positions 148 and 150 have only one orientation, and subaperture positions 152 and 154 have two orientations.

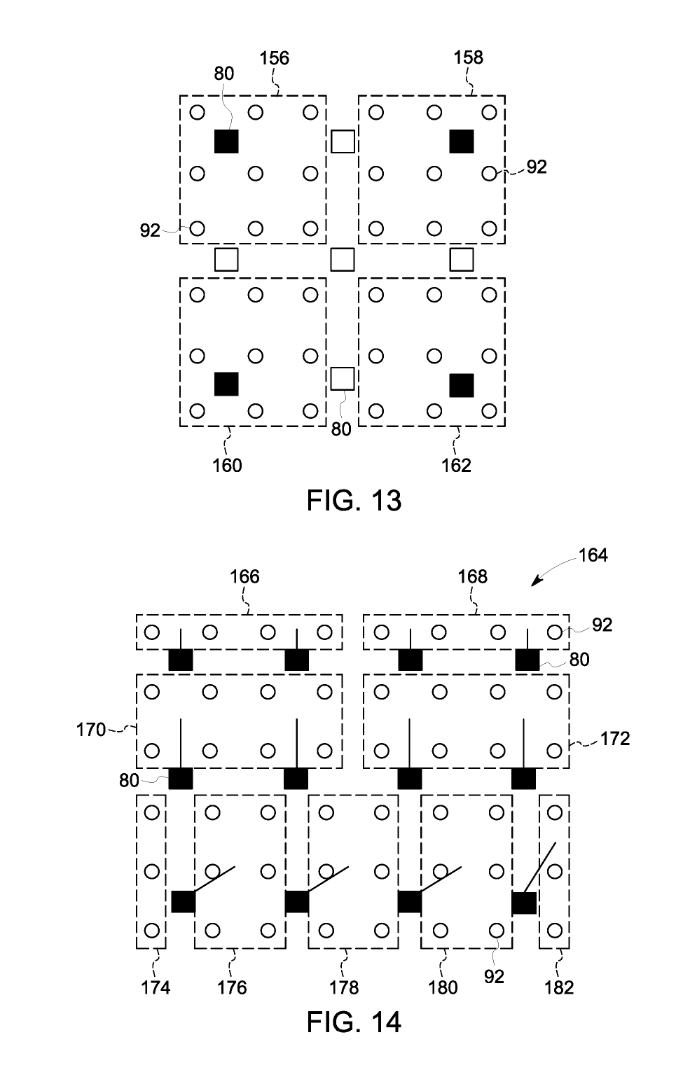

From FIG. 13, which shows the four orientations of a 3-by-3 subaperture, it should be clear that a subaperture for which both dimensions are three element nodes 92 or larger need use only internal summers 80 and, thus, has four orientations, as indicated by the subapertures labeled 156, 158, 160 and 162.

Advantageously, a table can be constructed which lists the orientations for each size of type-1 subaperture that will be used in a transducer aperture design, as discussed below. Each size of type-2 subaperture is defined by the number of its constituent subapertures and the sizes and orientations of its constituent subapertures.

### Aperture Tiling and Routing

As used herein, “tiling” refers to the subdivision of a two-dimensional region into a set of smaller regions, not necessarily of the same size. Preferably, apertures may be subdivided without gaps or overlapping subapertures, though this is not required. In the discussion that follows, tilings having no gaps or overlaps are described. As will be appreciated, such tilings (without gaps or overlaps) are the most constrained and are, therefore, often the most difficult to construct. A tiling (without gaps or overlaps) of a rectangular aperture by smaller rectangular subapertures always exists. However, in constructing a tiling for an aperture, one must also ensure that each subaperture can be assigned a summer or summers, i.e., assigned an orientation, without conflict. As used herein, the tiling is referred to as having been “routed,” if each subaperture in a tiling has been assigned to one or more unique summers, as described further below.

It should be clear that there can be no conflict in routing a tiling that consists entirely of subapertures with internal summers. There is no guarantee, however, that a tiling which uses subapertures with external summers can be routed. FIG. 14 shows a simple example, representing an aperture 164 consisting of 8×6=48 element nodes 92, not a portion of a larger aperture. The two 1-by-4 subapertures 166 and 168 at the top border of the aperture 164 must connect to the (external) summers 80 below them. As a guide for the instant discussion, lines are drawn from an external summer 80 to the interior of the associated subaperture (e.g., 166 and 168). The orientations assigned to the top row of subapertures 166 and 168 means there is no choice in the orientations of the second row of 4-by-2 subapertures 170 and 172; they must connect to the summers 80 below them. This leaves only four unassigned summers 80 for the five subapertures 174, 176, 178, 180 and 182 in the bottom row. Disadvantageously, this tiling of this aperture is “unroutable.”

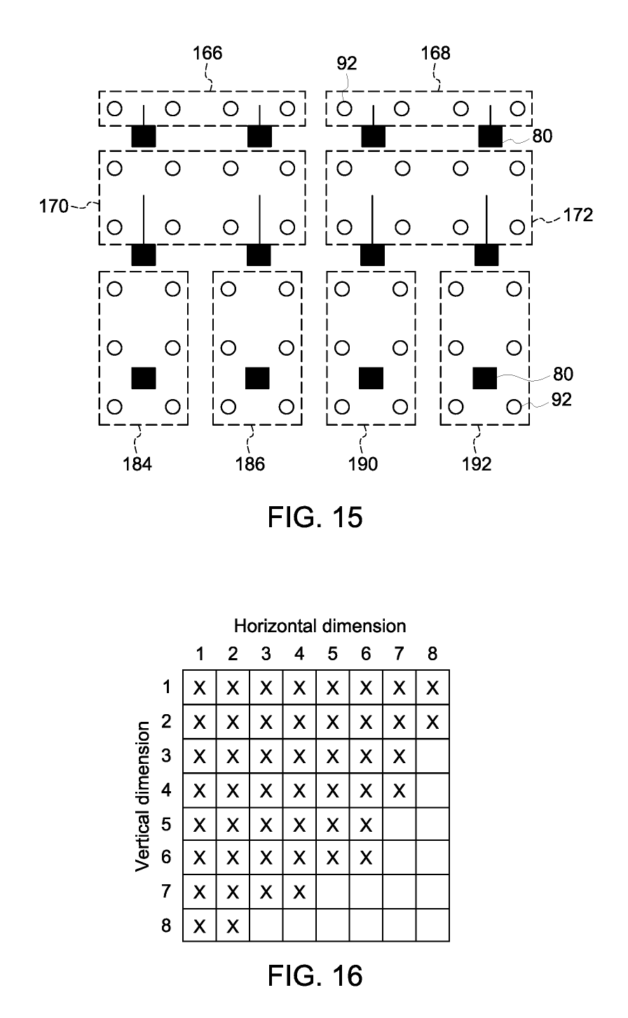

In some cases, a tiling can be routed with the proper choice of orientation of a subaperture or subapertures which have more than one possible orientation. In other cases, a tiling can be modified to make it routable. For example, the tiling shown in FIG. 14 can be routed by replacing the bottom row of 1-by-3 and 2-by-3 subapertures 174, 176, 178, 180 and 182 with four 2-by-3 subapertures 184, 186, 190 and 192, as shown in FIG. 15. Thus, the tiling in FIG. 15 is routed, since each subaperture in the tiling is assigned to a unique summer or summers 80. In this example, each of the subapertures 184, 186, 190 and 192 utilizes a respective internal summer 80.

### Aperture Tiling Examples

The disclosed architecture supports a wide variety of aperture tilings. The remaining examples are constructed using the set of type-1 subapertures with the dimensions indicated by “X” in the table shown in FIG. 16. One consideration in choosing a set of subaperture dimensions is that the maximum delay a first-level summer (e.g. summer 80) must support is roughly proportional to the length of the longest diagonal of the set of transducer elements connected to the element nodes of any type-1 subaperture. Larger, type-2 subapertures are constructed by combining two or four subapertures from this set. For example, a 12-by-12 type-2 subaperture can be constructed by summing, with delay, the outputs of four 6-by-6 constituent subapertures in a second-level summer (e.g., summer 114).

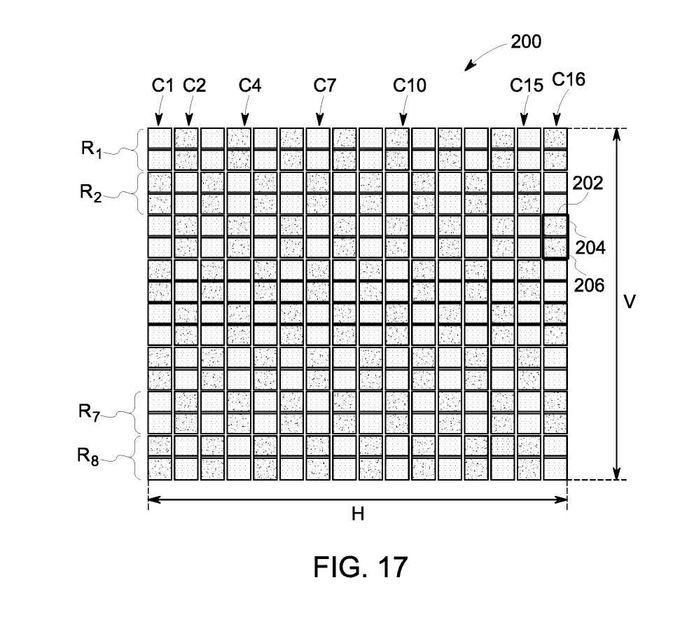

As an example of the flexibility of the architecture, FIG. 17 illustrates a rectangular aperture subdivided into equal-size subapertures. The aperture 200 is 96 elements wide (horizontal dimension H) and 80 elements tall (vertical dimension V). It is tiled using 128 equal-size subapertures, arranged in sixteen columns (C1-C16) and eight rows (R1-R8). Each subaperture is a 6-by-10, type-2 subaperture constructed from a pair of 6-by-5 subapertures stacked vertically. For illustrative purposes, one 6-by-10, type-2 subaperture 202, constructed from a pair of 6-by-5 subapertures 204 and 206, is marked in FIG. 17. The constituent 6-by-5 subapertures (e.g., subapertures 204 and 206) are drawn as rectangles, with each type-2 subaperture (e.g., subaperture 202) shaded. The shading of each type-2 subaperture (e.g., subaperture 202) alternates between light and dark for adjacent type-2 subapertures, for illustrative purposes. If each type-2 subaperture (e.g., subaperture 202) were connected to a separate system channel, this aperture 200 would average 60 elements per channel.

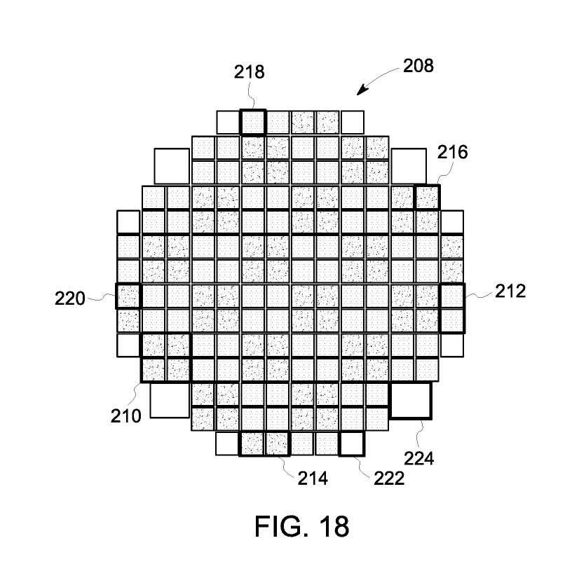

As another example of the flexibility of the architecture, FIG. 18 illustrates an embodiment of a roughly circular aperture. The aperture 208 consists of 2,576 elements contained within a 56-by-56 element square (not shown). It is tiled using 12 type-1 subapertures and 40 type-2 subapertures. The type-2 subapertures have dimensions 8-by-8 (e.g., subaperture 210), 4-by-8 (e.g., subaperture 212) and 8-by-4 (e.g., subaperture 214). These are constructed from 4-by-4 subapertures (e.g., subapertures 216, 218 and 220). The light or dark shading indicates the type-2 subapertures (e.g., subapertures 210, 212 and 214). The type-1 subapertures have dimensions 4-by-4 (e.g., subaperture 222) and 6-by-6 (e.g., subaperture 224). The type-1 subapertures (e.g., subapertures 222 and 224) are drawn as unshaded squares. As will be appreciated, this aperture tiling averages 49.5 elements per channel.

### Translatable Aperture Tiling

One advantageous feature described herein in accordance with embodiments of the invention is the ability to create “translatable” aperture tilings. These are tilings designed to have smaller subapertures near a “beam center” and larger subapertures farther away, with the total number of type-1 and type-2 subapertures, i.e., the number of required system channels, nearly constant. The “beam center” in ultrasound receive beamforming is a reference point corresponding in some sense to the intersection of a line of receive focus with the transducer aperture surface. Generally, a smaller fraction of the total transducer aperture area is used when focusing close to the array along the line of focus, and a larger fraction when focusing at more distance points. For example, one might arrange to preserve an approximately_constant f-number with depth. The apertures of one-dimensional transducer arrays are typically subdivided into equal-size elements, and this increase of transducer aperture area is implemented by allowing more elements to contribute to the receive beamsum when focusing at larger ranges. With equal-size elements, this scheme of adding elements to the beamsum with depth is easily implemented no matter where the beam center is positioned on the transducer aperture.

This method has a logical extension for two-dimensional transducer arrays subdivided into equal-size subapertures, as illustrated in FIG. 17. With subaperture beamforming, however, the acoustic directivity of the subapertures can become a limiting factor. The directivity of a focused subaperture is narrower than the directivity of its constituent elements (just as the directivity—the focus—of a transducer array is narrower than the directivity of its constituent transducer elements). In some circumstances, it would be desirable to use smaller subapertures, with wider directivity, when focusing close to the transducer array, while still retaining the property of increasing the active aperture with depth by adding subapertures to the beamsum. To support such a scheme, it can be beneficial to subdivide an aperture using subapertures whose sizes increase with distance from a beam center. A consideration in designing such tilings is that the number of subapertures in the tiling should be roughly constant, equal to the number of signals which are to be brought back to the console for further processing.

Such tilings can be found by trial-and-error, but this is a tedious and time-consuming process that must be repeated for each desired beam center. While one might need to define on the order of 100 beam centers for a one-dimensional transducer array, for a two-dimensional transducer array one might need to define of order 50×50=2,500 beam centers. Designing a few thousand such tilings by trial-and-error is a major undertaking. It is highly desirable to be able to construct translatable tilings algorithmically.

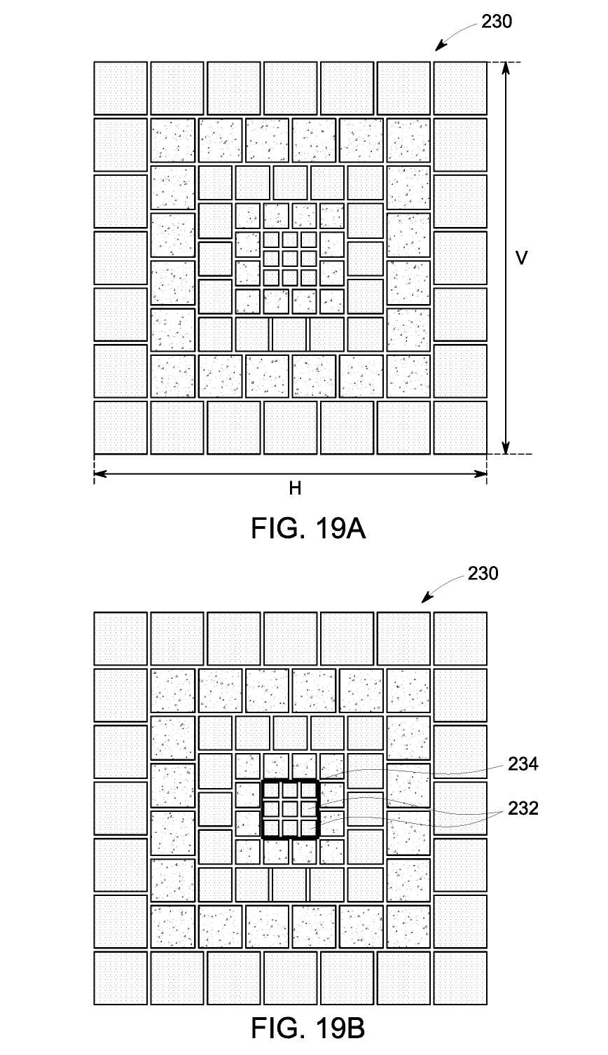

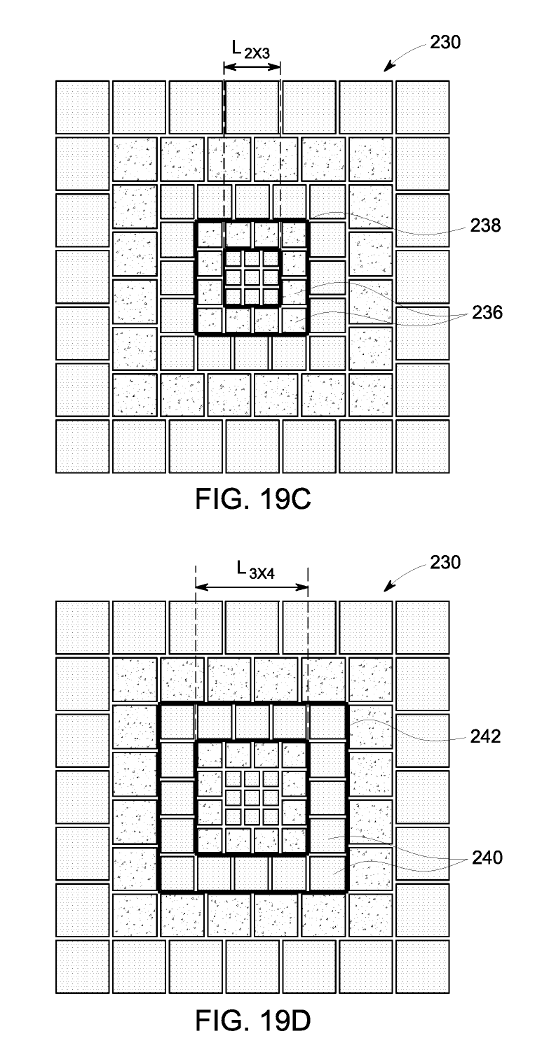

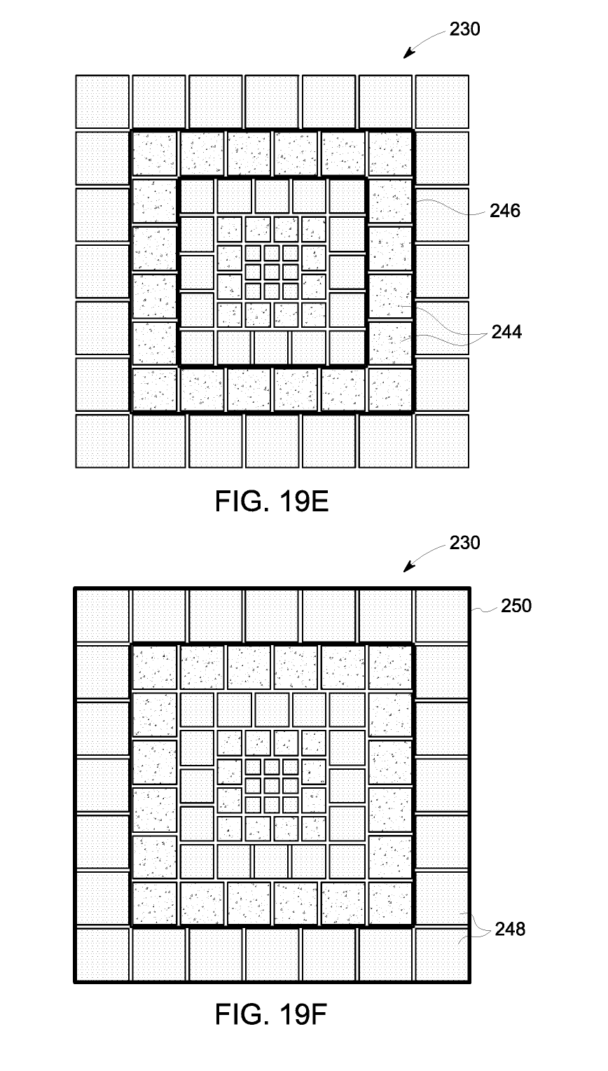

FIG. 19A illustrates a component of a translatable aperture constructed using the techniques disclosed in accordance with embodiments of the invention. The illustrated aperture 230 is a square aperture 42 elements wide, as described below. The aperture 230 is tiled using square subapertures with widths 2, 3, 4, 5 and 6 elements to form a “central region” formed from square subapertures arranged concentrically about one another, thus forming “concentric square rings.” As used herein, the “central region” of an aperture refers to the square portion of any aperture (e.g., aperture 230) formed of groups of square subapertures of various sizes, arranged in concentric square rings about the smallest innermost square of subapertures (e.g., 2-by-2 subapertures), as described in detail below. The central region of the aperture includes the largest square of the concentric square rings of square subapertures, and thus the central region is a square of subapertures having equal vertical (V) and horizontal (H) dimensions. Thus, in the embodiment illustrated in FIG. 19A, the entire aperture 230 forms a central region because the entire aperture 230 is a square formed by square subapertures surrounded by a number of concentric square rings of square subapertures. Each concentric square of subapertures is formed of subapertures of increasing size from the innermost portion of the central region to the outermost portion of the central region, as described below.

As illustrated, the smallest subapertures are in the center of the aperture 230, and the subaperture size increases with increasing distance from the center of the central portion of the aperture 230. This pattern of subaperture sizes is constructed by the following methodology: Nine 2-by-2 subapertures 232 are placed in three rows of three columns in the center of the aperture 230, as illustrated by the square 234 in FIG. 19B. These subapertures 232 are bordered by twelve 3-by-3 subapertures 236, forming a concentric square ring 238, surrounding the square 234 of nine 2-by-2 subapertures 232, as illustrated in FIG. 19C. This concentric square ring 238 of subapertures 236 is bordered by sixteen 4-by-4 subapertures 240, forming a larger concentric square ring 242, as illustrated in FIG. 19D. The larger concentric square ring 242 of subapertures 240 is bordered by twenty 5-by-5 subapertures 244, forming a larger concentric square ring 246, as illustrated in FIG. 19E. The larger concentric square ring 246 of subapertures 244 is bordered by 24 6-by-6 subapertures 248, forming a larger concentric square ring 250, as illustrated in FIG. 19F. This process of adding equal-sized subapertures (e.g., 232, 236, 240, 244 and 248) to the boundary to form a larger square can continue indefinitely, because of the following elementary arithmetic identities:

3×2=2×3

4×3=3×4

5×4=4×5

To see this, consider the top row of the group of two-element-wide subapertures 232 (FIG. 19B). This row has three subapertures 232. To create a second row above this, note that two three-element-wide subapertures 236 (FIG. 19C) are the same length L_{2×3} as three two-element-wide subapertures 232, since 3×2=2×3. Adding a three-element-wide subaperture 236 on either end completes the second row, which consists of four three-element-wide subapertures 236. Clearly the entire boundary of the width-two subapertures 232 can be completed with width-three subapertures 236 forming a concentric square ring 238 of subapertures 236.

To create a third row, note that three four-element-wide subapertures 240 (FIG. 19D) have the same length L_{3×4} as four three-element wide subapertures 236, since 4×3=3×4. Adding a four-element-wide subaperture 240 at either end completes the row, which consists of five four-element-wide subapertures 240. The entire boundary of three-element wide subapertures 236 can be surrounded by four-element wide subapertures 240 to form a larger concentric ring 242 of subapertures 240.

This process can be continued indefinitely. The number of subapertures in such an aperture is given by:

9+4×3+4×4+4×5+ . . . +4×p=9+2(p+3)(p−2),

where p is the width (and height) of the largest subaperture used. The aperture is p (p+1) elements wide, so that the average number of elements in the subaperture is

p²(p+1)²/[9+2(p+3)(p−2)].

For the example shown in FIGS. 19A-19F, p=6, and the aperture 230 is 42 elements wide. The aperture 230 includes of 81 subapertures which increase in size further from the center of the aperture 230 and contains an average of about 21.8 elements per subaperture. As previously described, the arrangement of subapertures can be referred to as “concentric square rings” of square subapertures. That is, at the center of the aperture 230, a square 234 is formed by the 2-by-2 subapertures 232. A concentric square ring 238 of 3-by-3 subapertures 236 is formed around the square 234. A concentric square ring 242 of 4-by-4 subapertures 240 is formed around the concentric square ring 238. A concentric square ring 246 of 5-by-5 subapertures 244 is formed around the concentric square ring 242. Finally, a concentric square ring 250 of 6-by-6 subapertures 248 is formed around the concentric square ring 246.

It will be appreciated that the size of the subapertures in the tiling of FIGS. 19A-19F increases approximately with distance from the center of the aperture 230, which is the center of the innermost 2-by-2 subaperture. Thus this aperture and its tiling is a useful component of a larger, translatable aperture tiling designed to acquire a beam with a beam center at, or near, the center of the transducer elements connected to the innermost 2-by-2 subaperture.

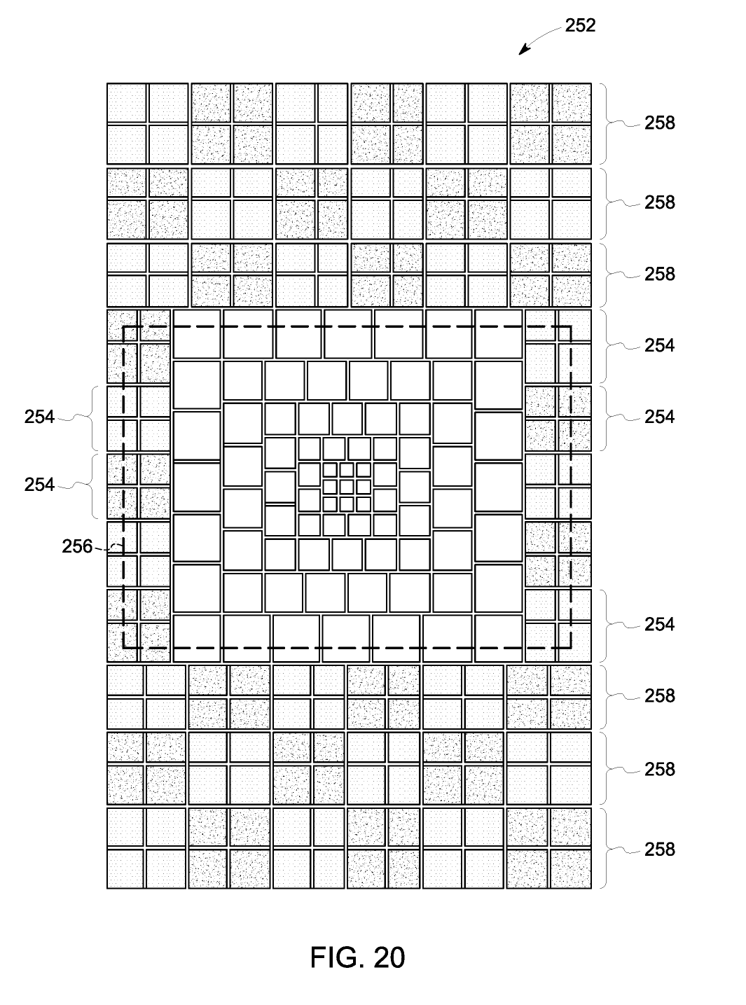

FIGS. 20, 21 and 22 illustrate an example of a second component of a translatable aperture tiling, referred to herein as a “vertical core.” In the case illustrated in FIGS. 20, 21 and 22, the vertical core is a 58-by-96-element aperture 252 divided into 127 subapertures. In FIG. 20, the type-1 subapertures are unshaded, for illustrative purposes, and the shading of each type-2 subaperture alternates between light and dark for adjacent type-2 subapertures. The constituent type-1 subapertures for the type-2 subapertures are drawn as rectangles. The tiling includes the 42-by-42-element central region of FIGS. 19A-19F. This central region (unshaded portion of FIG. 20) is centered both horizontally and vertically in the aperture 252. The central region is bracketed on either side by a column of five type-2 subapertures 254, each 8 elements wide and with heights 9, 8, 8, 8, and 9 elements. This forms a rectangular region, referred to as a “core 256,” 56 elements wide and 42 elements high in this case, indicated by the dashed line passing through the outermost subapertures of the core 256. As used herein, the “core” (e.g., core 256) includes the central region (e.g., the aperture 230, which in its entirety is a central region, as described above) and the columns of subapertures 254 on either side of the central region 230 and extending horizontally to the outer edges of the vertical core 252 (e.g., the columns of subapertures 254 on either side of the central region 230 and extending horizontally to the outer edges of the vertical core 252). In FIG. 20, the core 256 is centered vertically in the aperture 252. Three rows of type-2 subapertures 258 lie above and three rows of type-2 subapertures 258 lie below the core 256, with heights of 8, 9 and 10 elements; the widths are 10, 10, 9, 9, 10 and 10 elements. The core 256 and the surrounding rows of subapertures 258 form the “vertical core,” which in this example includes 127 subapertures (81 type-1 subapertures, shown unshaded in FIG. 20, and 46 type-2 subapertures, shown as alternating light and dark shaded portions of FIG. 20).

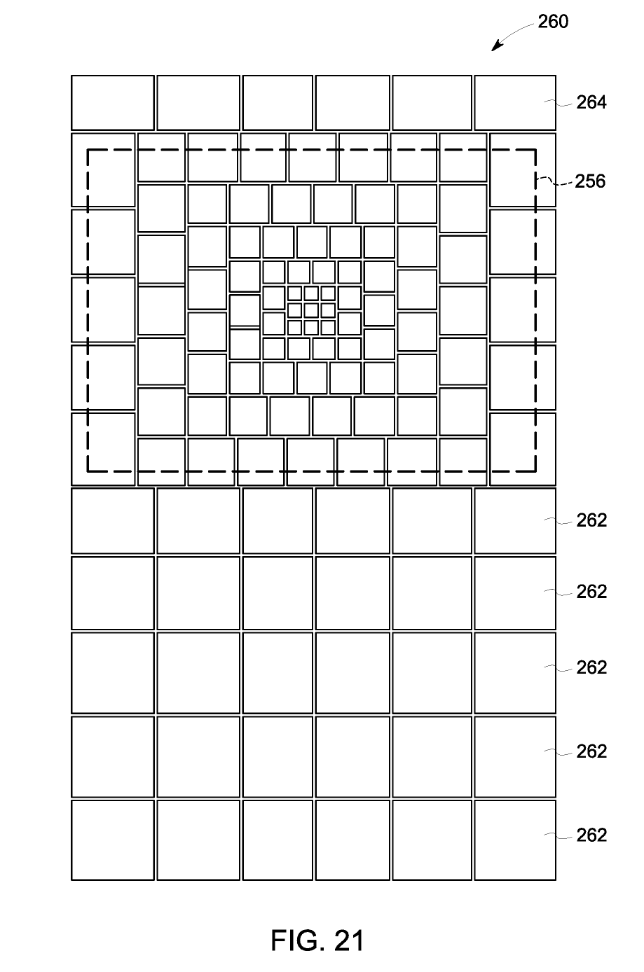

FIG. 21 shows another tiling of a 58-by-96-element aperture 260 which uses the same number of subapertures as that in FIG. 20 but with the core 256 (dashed line) translated vertically by 20 elements from the aperture center. The constituent subapertures of the type-2 subapertures are not shown in this figure; the rectangles indicate the type-1 and type-2 subapertures used in the tiling. The tiling in FIG. 21 differs from that in FIG. 20 only in the number and heights of the rows of subapertures above and below the core. There are five rows of subapertures 262 below the core 256, with heights 8, 9, 10, 10 and 10 elements, and one row of subapertures 264 above the core, of height 7 elements.

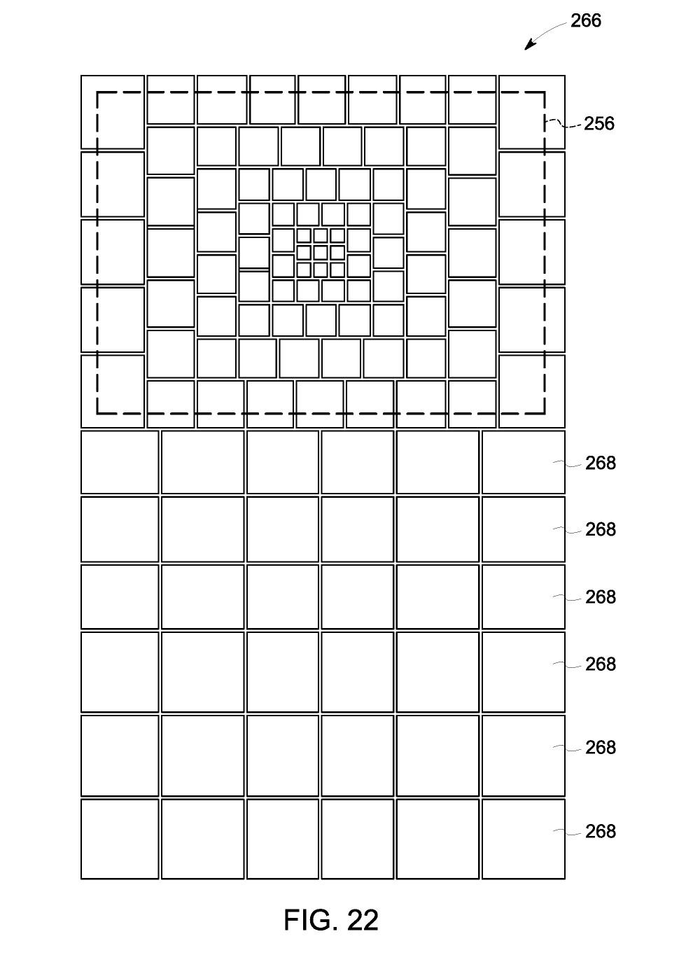

FIG. 22 shows yet another tiling of a 58-by-96 element aperture 266 which uses the same number of subapertures as that shown in FIGS. 20 and 21. The core 256 (dashed line) is translated vertically by 26 elements with respect to the aperture center. There are six rows of subapertures 268 below the core 256, with row heights 8, 8, 8, 10, 10 and 10, and no rows of subapertures above the core 256. It will be appreciated that the tiling of the core 256 and the total number of rows above and below the core is the same in FIGS. 20, 21 and 22.

In FIGS. 20, 21 and 22, the core 256 translates vertically, and six horizontal rows are distributed above and below the rectangular core 256. The row heights and the distribution of rows above and below the core are chosen so that the row heights are roughly equal (e.g., 8-10 elements high), with smaller row heights used for rows which are closer to the core 256. A tiling for any desired vertical translation from the aperture center of the core 256 by an integer number of elements can be constructed in this way, by specifying the heights of each of the six rows and by specifying how many of the six rows lie below the core 256. It will be appreciated that this method of designing a set of vertically translatable apertures is considerably less complex than the general problem of dividing, without gaps or overlaps, the 58-by-96 element aperture 266 into 127 subapertures whose size increases approximately with distance from a set of specified vertical positions of a beam center.

An important feature of this disclosed method of tiling is that the tiling has the same number of subapertures independent of the vertical translation of the beam center. This is desirable since the number of subapertures should be close to, but never larger than, the number of system channels. Another important feature is that for a given aperture size, the tiling of the core and the widths of the subapertures in the rows used to fill the aperture above and below the core need only be chosen once. Then the tiling for any vertical translation of the core from the aperture center is completely specified by seven additional numbers: the number of rows of subapertures used below the core, and the height of each of the six rows of subapertures used above and below the core. This scheme of constructing a class of vertically translating apertures is advantageously simpler than, for example, trying to construct a tiling for a fixed number of subapertures without any constraints.

The largest vertical translation of the core from the aperture center which can be generated using this method corresponds to placing the upper border of the core at the upper border of the aperture, as in FIG. 22. If desired, apertures with the center of the core closer to the aperture boundary can be created by shrinking one or more rows of the core.

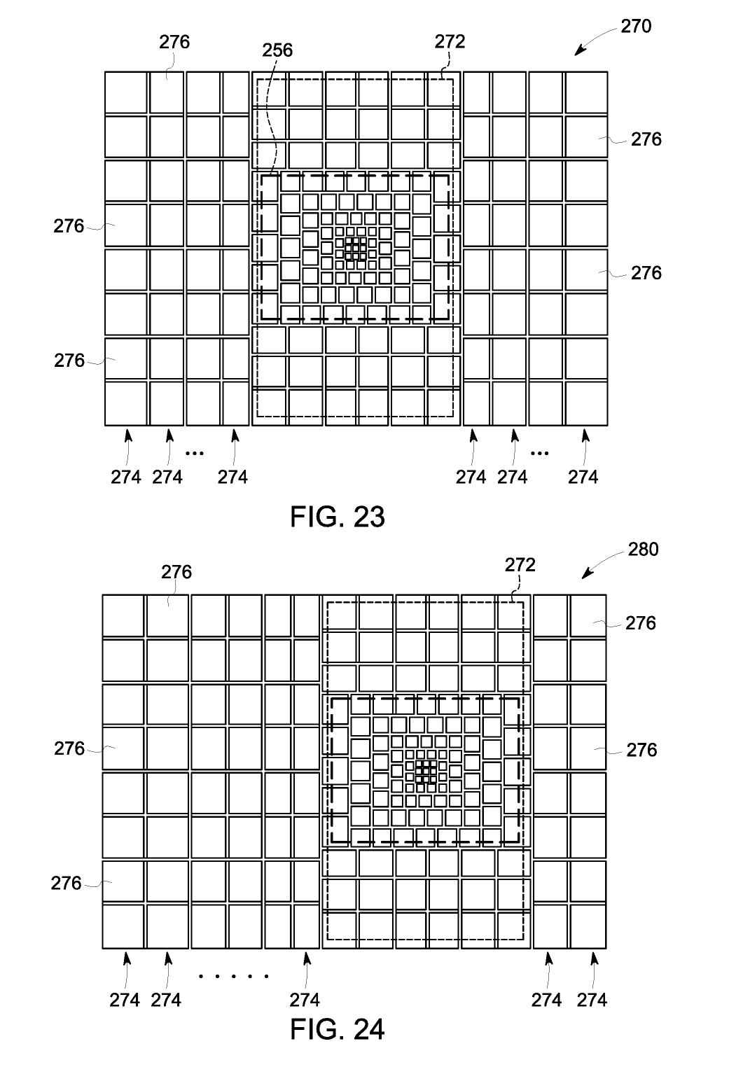

A similar method is used to allow for horizontal translations of the core. FIG. 23 shows a 138-by-96 element aperture 270 tiled using 191 subapertures (81 type-1 and 110 type-2 subapertures). A dotted line is drawn through the outermost subapertures of the 56-by-96 element vertical core 272 (shown as vertical core 252 in FIG. 20). A dashed line is drawn through the outermost subapertures of the 56-by-42 element core 256. There are four columns 274 of eight subapertures 276 on either side of the vertical core 272. The columns 274 to the left of the vertical core 272 have widths 12, 10, 10 and 8 and the columns 274 on the right of the vertical core 272 have widths 8, 10, 10 and 12. The eight subapertures 276 in each column 274 are all 12 elements high.

FIG. 24 shows a 138-by-96 element aperture 280 in which the vertical core 272 is translated to the right by 20 elements from the aperture center. This tiling also utilizes 191 subapertures 276. There are again eight columns 274 of subapertures bracketing the vertical core 272, six on the left, with widths 12, 12, 10, 10, 8 and 8, and two on the right, with widths 10 and 10.

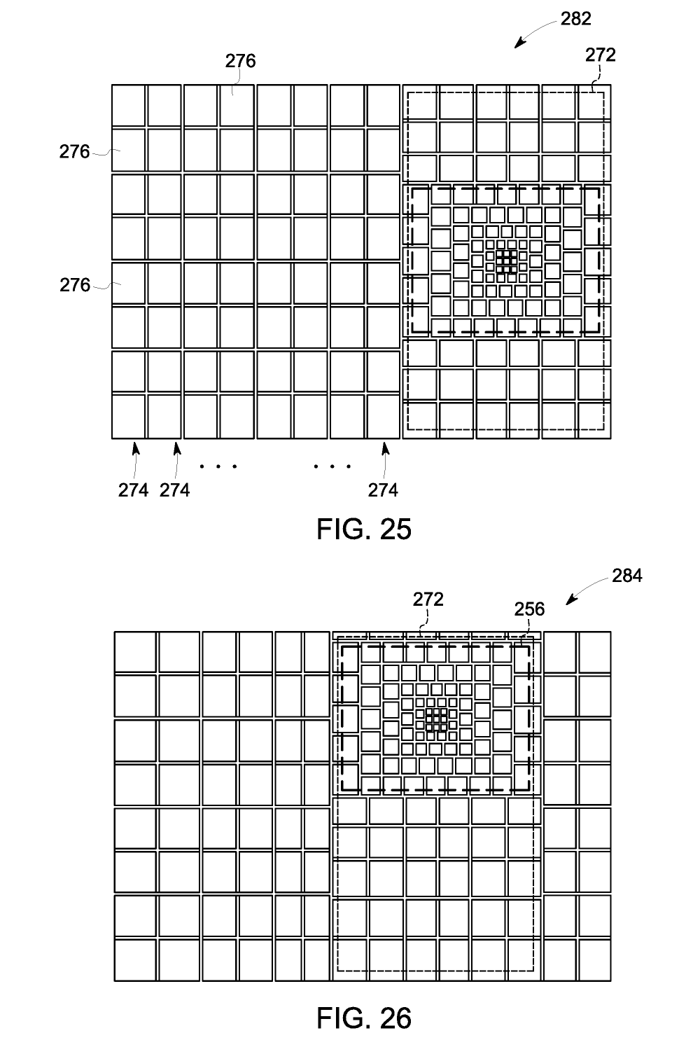

In FIG. 25 the vertical core 272 is translated to the right by 40 elements from the aperture center. There are eight columns 274 of subapertures 276 to the left of the vertical core 272, each of width 10 elements and no columns on the right. This tiling also uses 191 subapertures 276.

FIGS. 23, 24 and 25 illustrate a method of designing apertures (e.g., apertures 270, 280 and 282) which are tiled with a fixed number of subapertures 276 and in which the core can be translated horizontally by a specified number of elements. In this example, eight columns 274, each consisting of eight subapertures 276, each having a height of 12 elements, are placed on either side of a vertical core 272. The column widths and the distribution of columns 274 to the left and to the right of the vertical core 272 are chosen so that the column widths are roughly equal, with smaller columns widths used for rows which are closer to the vertical core 272. A tiling for any desired horizontal translation from the aperture center of the vertical core 272 by an integer number of elements can be constructed in this way, by specifying the widths of each of the eight columns and by specifying how many of the eight columns lie to the left of vertical core

FIG. 26 illustrates that core can be translated both horizontally and vertically within the 138-by-96 element aperture 284 by combining a vertical translation of the core 256 with a horizontal translation of the vertical core 272. In FIG. 26 the core 256 has been translated vertically by 25 elements and the vertical core has been translated horizontally by 24 elements.

It will be appreciated that this design decomposes a two-dimensional translation of the core into a pair of one-dimensional translations. This reduces the number of aperture tilings which must be designed and specified from H×V, where H is the number of desired horizontal translations and V is the number of desired vertical translations, to H+V. For example, if H=25 and V=25, this method advantageously reduces the number of aperture tilings which must be designed by an order of magnitude, from 625 to 50. Moreover, as has been previously explained, this design reduces the number of parameters which must be specified for the tiling for any given horizontal and vertical translation of the core. For the example aperture 284, instead of requiring the specification of the heights and widths of 191 subapertures, in our design only sixteen numbers are specified: the heights of six rows, the widths of eight columns, the number of rows below the core, and the number of columns to the left of the core. Furthermore, finding values of those sixteen numbers which produce a tiling with no gaps or overlaps is straightforward; in contrast, finding the heights and widths of 191 subapertures with the same property is non-trivial.

### Multiple ASIC Design

The apertures illustrated in FIGS. 23, 24, 25 and 26 are tiled using 191 subapertures and, thus, are designed for an ultrasound imaging system with at least 191 system channels. While a single ASIC could be used for the circuitry, switches and wiring to implement the subaperture processing for these tilings, it is often simpler and less expensive to design a single ASIC to support fewer channels and to use multiple ASICs, as needed, for a given probe design. This simplifies the ASIC design because, for example, fewer signal and control lines need to be routed. The overall system cost is usually less because the manufacturing yield will be higher for a smaller ASIC. The number of transducer elements and system channels is often different for different imaging applications. A 2D cardiac probe might be designed for 64×64 elements, for example, while a 2D abdominal probe might be designed for 192×64 elements. Designing a separate ASIC for each application would typically be considerably more expensive than designing a single, smaller ASIC that could be used as a modular component for both probes.

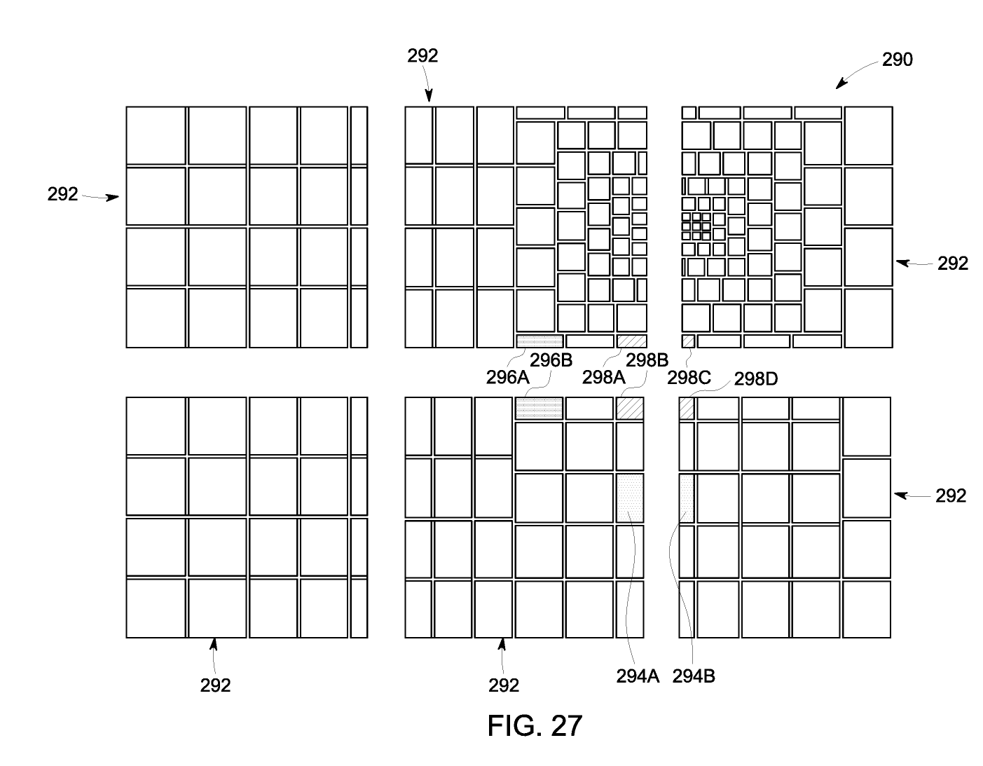

Dividing an aperture tiling among multiple ASICs introduces a new consideration. FIG. 27 shows a 138-by-96 element aperture tiling with the beam center translated horizontally by 30 elements and vertically by 24 elements. The aperture 290 has been divided into six 46-by-48 element apertures 292, which are referred to herein as “ASIC apertures.” An ASIC aperture refers to a spatial arrangement of element nodes coupled to a particular ASIC. As will be appreciated, the subapertures on each “ASIC aperture” may be controlled by a single ASIC. That is, a single aperture (e.g., aperture 290) may be divided into additional apertures (e.g., ASIC apertures 292), wherein each of the ASIC apertures 292 is controlled by a single ASIC. For clarity, FIG. 27 leaves a small gap between the six ASIC apertures 292. In general, the ASIC aperture boundaries will not lie along the subaperture boundaries in the original, undivided tiled aperture. When this happens, the subaperture of the original tiling which straddles an ASIC boundary is divided into either two or four subapertures: two subapertures, if the subaperture straddles a horizontal or vertical boundary between two ASIC apertures 292, and four subapertures, if the subaperture straddles the corner where four ASIC apertures 292 meet. The light shading in FIG. 27 marks an example of a subaperture split into two subapertures 294A and 294B at a vertical boundary. The dark shading marks an example of a subaperture split into two subapertures 296A and 296B at a horizontal boundary. The hatching marks an example of a subaperture split into four subapertures 298A, 298B, 298C and 298D at a corner. These subaperture splittings are easily generated algorithmically. The subapertures created by splitting a subaperture at an ASIC aperture boundary are ultimately connected to the same system channel.

The disclosed architecture supports these aperture tilings and a wide variety of others. The block diagram illustrated in FIG. 10, has Q outputs, and therefore can form Q subapertures, of which at most P can be type-2 subapertures. The values P and Q can be determined by exhaustive search of all desired apertures for a given transducer array, with the apertures optionally split into more than one ASIC apertures. The value P should be no smaller than the maximum number of type-2 subapertures in any ASIC aperture. The value Q should be a value no smaller than the maximum number of type-1 and type-2 subapertures in any ASIC aperture.

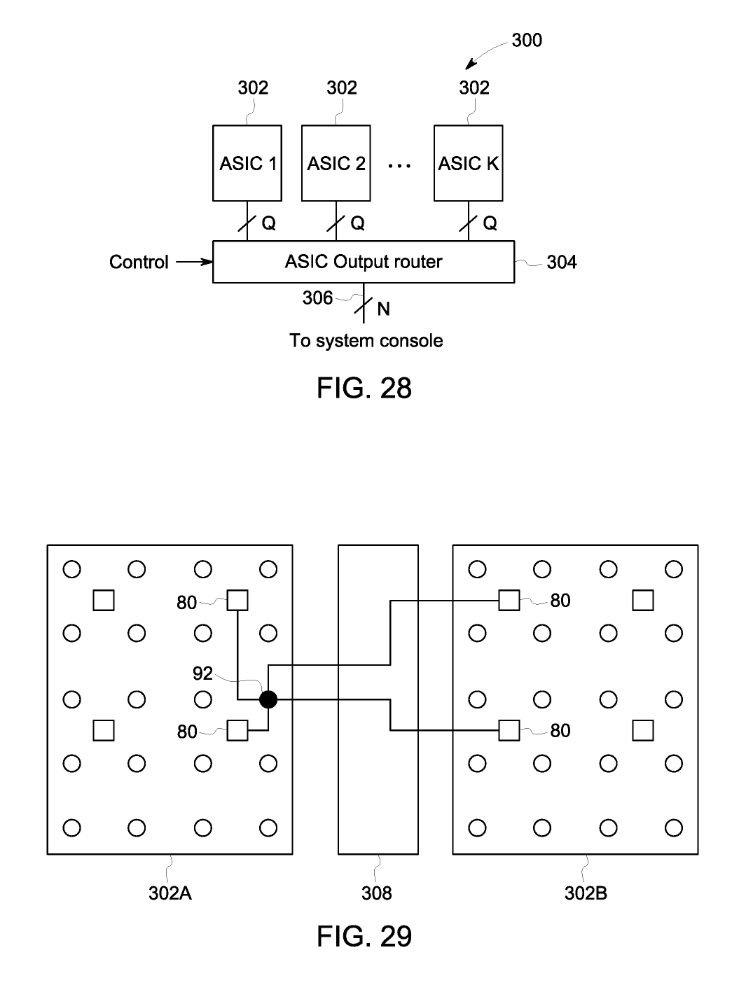

FIG. 28 illustrates a partial block diagram of receive beamforming electronics 300 for a two-dimensional probe for use in an ultrasound imaging system, wherein multiple ASICs 302 are employed. That is, multiple discrete silicon chips having integrated circuits formed on a substrate (e.g., silicon substrate) are utilized. Each ASIC 302 supports the subaperture processing for a portion of the probe's transducer elements, as previously described. If K ASICs 302 are used for the probe, there will be K×Q outputs from the set of ASICs 302, as shown in FIG. 28. L will be the largest number of type-1 and type-2 subapertures in any desired aperture tiling for the probe's transducer array. When K is one, i.e., when the probe uses only a single ASIC 302, then the aperture tilings will typically be designed so that the number of subapertures, L, is equal to or slightly less than the number of system channels, N. When K is greater than 1, and especially when translatable aperture tilings are used, K×Q will typically be larger than L, i.e., there will be more ASIC outputs Q than subapertures. The reason for this is that for many types of tilings, such as the class of translatable apertures, the number of subapertures is not the same in every ASIC aperture, as FIG. 27 illustrates. The number of ASIC outputs Q should be at least as large as the largest number of subapertures in any ASIC aperture. In other words, an ASIC 302 must support more than the average number of subapertures, i.e., Q will be larger than L/K, which is equivalent to K×Q being larger than L.

A second level of signal routing may then be employed, as shown in FIG. 28, to reduce the K×Q outputs from the K ASICs 302 to L signals which are sent, via a probe cable or wireless link, to the N beamforming channels on the system console. The aperture tiling is designed so that L is less than or equal to N; if L is less than N, then only L of the N system channels will be used. No relative time-delay is imposed on the inputs to the block element labeled “ASIC Output Router 304” in FIG. 28. A relatively small number of signals from different ASICs 302—those signals from subapertures that were formed by splitting a subaperture which straddled an ASIC aperture boundary (as described in FIG. 27)—are summed (without delay) in the ASIC Output Router 304 to a common output 306. FIG. 28 places the ASIC Outer Router 304 block before the cable connecting the probe to the system console, since this reduces the number of signals which must be transmitted to the system console through the cable (or through a wireless connection). However, it will be appreciated that the ASIC Output Router 304 could be placed after the transducer cable or wireless connection, if desired.

When more than one ASIC 302 is used, as in FIG. 28, the routing layer, e.g., flexible circuit or interposer, that is used to connect transducer elements to the element nodes on the ASIC 302 can also be used to connect elements near an ASIC aperture border to first-level summers on adjacent ASICs 302. This is illustrated in the top view provided in FIG. 29, which illustrates the topology of the signal connections between element nodes and summers provided on multiple ASICs. Specifically, FIG. 29 illustrates one element node 92 (filled circle) on the border of a first ASIC 302A and the four first-level summers 80 to which it can connect. Two of these summers 80 lie on the first ASIC 302A, and two of these summers 80 lie on a second ASIC 302B. The connections to the latter summers 80 are made using the routing layer 308, which can be an interposer layer or alternatively, a flexible circuit. Making these connections for every element node 92 on the interior ASIC boundaries means that the element nodes 92 and first-level summers 80 for all ASICs 302 (e.g., 302A and 302B) in a multiple-ASIC probe design are connected as if they were implemented on a single, larger ASIC. This eliminates the need to split subapertures at the ASIC borders, such as was illustrated in FIG. 27.

As described herein, the routing layer 308 may be an interposer. As will be appreciated, as used herein, an interposer refers to a layer arranged vertically between the transducer elements and the ASICs. That is, the transducer elements are physically located on one side of the interposer and the ASICs are located on a second side of the interposer, wherein the interposer provides signal paths from one side of the interposer to the other side of the interposer, in order to electrically couple the transducer elements to the ASICs. At least a portion of the transducer elements and the ASICs will be arranged vertically above or below one another on opposite sides of the interposer. Alternatively, the routing layer 308 may be a flexible circuit. As with the interposer, the flexible circuit electrically couples the transducer elements to the ASICs. The transducer elements may be arranged on one side of the flexible circuit. In some embodiments, the ASICs may be arranged on the same side of the flexible circuit as the transducer elements. In another embodiment, the ASICs and transducer elements may be arranged on opposite sides of the flexible circuit, but not arranged vertically with respect to one another for any portion of their geometries. That is, the ASICs may be arranged on an opposite side of the flexible circuit some distance down the length of the flexible circuit such that the transducer elements are not arranged vertically above any portion of the ASICs, for instance.

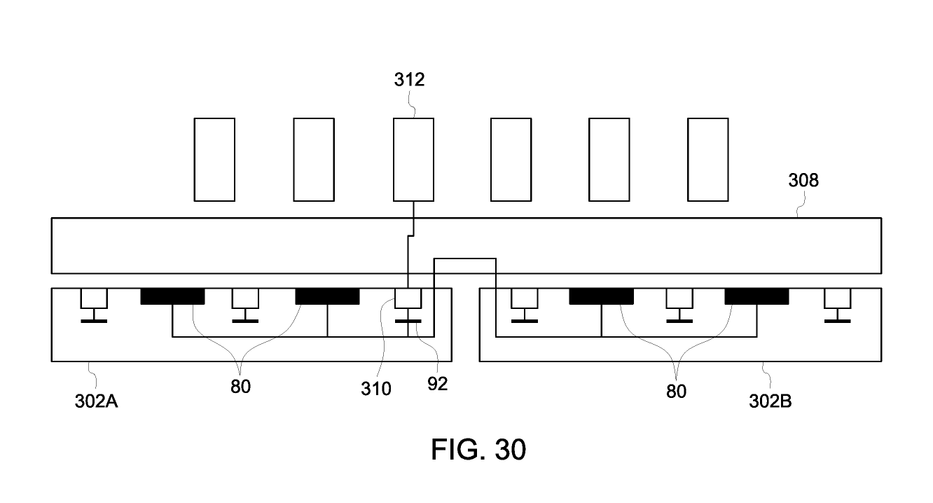

FIG. 30 illustrates a cross-section of the example described with regard to FIG. 29. As illustrated in FIG. 30, an element node 92 is connected to two summers 80 on a first ASIC 302A and connected to two summers 80 on a second ASIC 302B. The signal conditioning circuitry 310 (also referred to herein as signal conditioning circuit 310) corresponding to the illustrated element node 92 resides on the ASIC 302A. The transducer element 312 corresponding to the element node 92 is coupled to the signal conditioning circuitry through the interposer 308. By utilizing the interposer 308 (or alternatively, a flexible circuit), the element node 92 coupled to a given transducer element 312 may be switchably connected to summers 80 residing on an independent ASIC (here, ASIC 302B). Advantageously, making these connections for every element node 92 on the interior ASIC boundaries means that the element nodes 92 and first-level summers 80 for all ASICs 302 (e.g., 302A and 302B) in a multiple-ASIC probe design are connected as if they were implemented on a single, larger ASIC, thereby eliminating the need to split subapertures at the ASIC borders. Thus, the routing layer, shown as interposer 308 in this example, allows for some transducer elements 312 to be switchably connected to circuit elements (e.g., first-level summers 80) on two different ASICs 302A and 302B, simplifying the control of subapertures that extend past ASIC boundaries.

The cross-sectional representation of FIG. 30 illustrates a stacked arrangement, wherein the array of transducer elements 312 is arranged on top of the one or more ASICs 302A and 302B. In this vertically stacked arrangement, the interposer 308 is arranged between the array of transducer elements 312 and ASICs 302A and 302B. However, if, for example, a flexible circuit is utilized to provide the connection paths between transducer elements 312 and the ASICs 302A and 302B, the transducer elements 312 may not be located in a plane vertically above the ASICs 302A and 302B, as in the stacked arrangement. Instead, the transducer elements 312 may be arranged horizontally adjacent to the ASICs 302A and 302B. Further, in this arrangement, the transducer elements 312 may be located on the same side of the flexible circuit as the ASICs 302A and 302B, rather than on opposite sides, as in the stacked arrangement of FIG. 30.

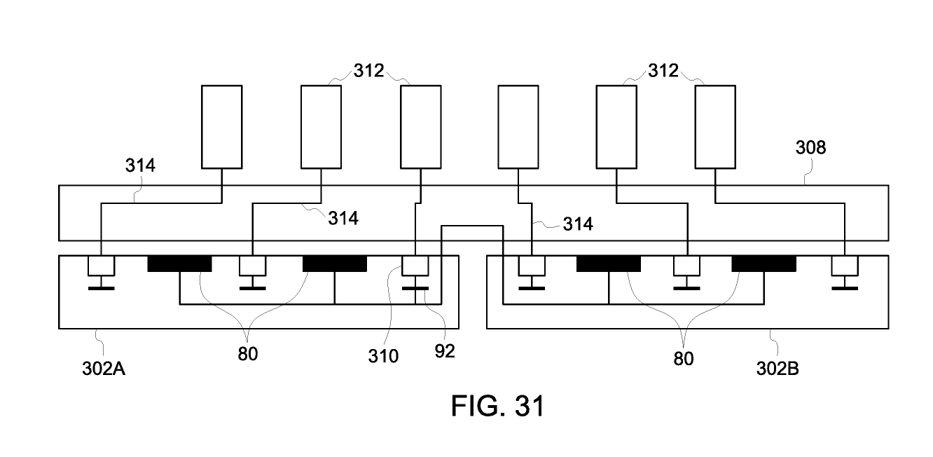

By employing a routing layer (e.g., a flexible circuit or an interposer 308) between the transducer elements 312 of the transducer array and the signal conditioning circuitry 310 for an element node 92 that may be fabricated on a single ASIC or different and discrete ASICs (e.g., ASICs 302A and 302B), the pitch of transducer elements 312 is decoupled from the pitch of the circuit elements, such as the signal conditioning circuitry 310. This advantage is illustrated in FIG. 31. As illustrated in the stacked configuration of FIG. 31, each transducer element 312 is electrically coupled to a respective signal conditioning circuit 310 through a respective signal path 314 formed through the interposer 308. As illustrated, the signal paths 314 may include at least a portion which is horizontal with respect to the stacked arrangement of the transducer elements 312 and the signal conditioning circuitry 310 of the element node 92. Notable in FIG. 31 is that the horizontal position of a transducer element 312 is not bound by the horizontal position of its respective signal conditioning circuitry 310, i.e., the transducer element 312 need not be positioned over its respective signal conditioning circuitry 310 Each signal path 314 allows for the decoupling of the pitch or horizontal spacing between the transducer elements 312 and the pitch of the respective signal conditioning circuitry 310 on an underlying ASIC 302A or 302B. In other words, the transducer element 312 does not have to be arranged vertically above, or within the vertical footprint of, the underlying signal conditioning circuitry 310. This allows for, for example, for more densely arranged transducer elements 312 with respect to the underlying circuitry on the ASICs 302A and 302B, and for one ASIC design to be used in more than one ultrasound probe design, which provides greater design flexibility and design and manufacturing cost savings. Thus, the pitch or horizontal spacing of the transducer elements 312 may be different from the pitch or horizontal spacing of the signal conditioning circuitry 310.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A transducer assembly, comprising:
a routing layer;
a plurality of transducer elements arranged on a first side of the interposer; and
a first application specific integrated circuit (ASIC) arranged vertically below the plurality of transducer elements and on a second side of the interposer, wherein the first ASIC comprises a plurality of signal conditioning circuits.

2. The transducer assembly of claim 1, wherein each of the plurality of signal conditioning circuits corresponds to a respective one of the plurality of transducer elements.

3. The transducer assembly of claim 1, wherein the routing layer comprises a plurality of signal paths configured to electrically couple each of the plurality of signal conditioning circuits corresponds to a respective one of the plurality of transducer elements.

4. The transducer assembly of claim 1, wherein a horizontal spacing of the plurality of transducer elements is different from a horizontal spacing of the plurality of signal conditioning circuits.

5. The transducer assembly of claim 1, wherein the routing layer comprises an interposer.

6. The transducer assembly of claim 5, wherein the interposer comprises a signal path to electrically couple a first of the plurality of transducer elements to a first of the plurality of signal conditioning circuits, wherein at least a portion of the signal path is horizontal.

7. The transducer assembly of claim 5, wherein the interposer comprises a signal path to electrically couple a first of the plurality of transducer elements to a first of the plurality of signal conditioning circuits, wherein the first of the plurality of transducer elements is not arranged directly above the first of the plurality of signal conditioning circuits.

8. The transducer assembly of claim 5, comprising a second ASIC arranged vertically below the plurality of transducer elements and on the second side of the interposer, wherein the second ASIC comprises a second plurality of signal conditioning circuits, wherein each of the plurality of signal conditioning circuits on the first ASIC corresponds to a respective one of a portion of the plurality of transducer elements, and wherein each of the second plurality of signal conditioning circuits on the second ASIC corresponds to a respective one of a second portion of the plurality of transducers.

9. The transducer assembly of claim 1, wherein the first ASIC comprises a plurality of summers switchably coupled to the plurality of transducer elements through the routing layer.

10. The transducer assembly of claim 8, comprising a second ASIC comprising a second plurality of summers switchably coupled to the plurality of transducer elements through the routing layer.

11. The transducer assembly of claim 10, wherein at least one of the plurality of transducer elements is coupled to at least one of the plurality signal conditioning circuits and to at least one of the plurality of summers on the first ASIC and at least one of the plurality of summers on the second ASIC.

12. A transducer assembly, comprising:
a first application specific integrated circuit (ASIC);
a second application specific integrated circuit (ASIC); and
a subaperture comprising a plurality of transducer elements, wherein a first portion of the subaperture is coupled to subaperture processing circuitry on the first ASIC and a second portion of the subaperture is coupled to subaperture processing circuitry on the second ASIC.

13. The transducer assembly of claim 12, wherein the subaperture processing circuitry comprises a first plurality summers on the first ASIC and a second plurality of summers on the second ASIC, wherein at least one of the plurality of transducer elements is coupled to at least one of the first plurality of summers and at least one of the second plurality of summers.

14. The transducer assembly of claim 12, wherein the first portion of the subaperture aperture processing circuitry comprises a first plurality of element nodes and corresponding signal conditioning circuitry, and wherein the second portion of the subaperture processing circuitry comprises a second plurality of element nodes and corresponding signal conditioning circuitry.

15. The transducer assembly of claim 12, wherein the first portion of the subaperture processing circuitry comprises a first plurality of summers and the second portion of the subaperture processing circuitry comprises a second plurality of summers.

16. The transducer assembly of claim 12, comprising a routing layer coupled to each of the first ASIC, the second ASIC and plurality of transducer elements, wherein the routing layer is configured to provide one or more signal paths from the plurality of transducer elements to each of the first ASIC and the second ASIC.

17. The transducer assembly of claim 12, wherein the plurality of transducer elements is arranged in a stacked configuration, vertically above the first ASIC and the second ASIC.

18. The transducer assembly of claim 17, comprising an interposer arranged vertically between the plurality of transducer elements and the first ASIC and second ASIC.

19. The transducer assembly of claim 12, wherein the subaperture is a type 1 subaperture.

20. A transducer assembly, comprising:
a plurality of transducer elements;
a first application specific integrated circuit (ASIC);
a second application specific integrated circuit (ASIC);
a routing layer communicatively coupled to the plurality of transducer elements and each of the first ASIC and the second ASIC.

21. The transducer assembly of claim 20, wherein the routing layer comprises an interposer.

22. The transducer assembly of claim 21, wherein the interposer is arranged in a vertical stack between the plurality of transducer elements and each of the first ASIC and the second ASIC.

23. The transducer assembly of claim 20, wherein at least one of the plurality of transducer elements is communicatively coupled to a first summer on the first ASIC and a second summer on the second ASIC.

24. The transducer assembly of claim 20, wherein the at least one of the plurality of transducer elements is communicatively coupled to a signal conditioning circuit on the first ASIC through a signal path of the routing layer.

25. The transducer assembly of claim 24, wherein the signal path comprises a vertical portion and a horizontal portion.

26. The transducer assembly of claim 20, wherein one of the plurality of transducer elements is arranged in a horizontal plane above a horizontal plane of the first ASIC and is electrically coupled to a signal conditioning circuit on the first ASIC.

27. The transducer assembly of claim 26, wherein the one of the plurality of transducer elements is not directly above the signal conditioning circuit.

28. The transducer assembly of claim 20, comprising a number of subapertures that is less than or equal to a number of beamforming channels of a console configured to receive sub aperture signals from the transducer assembly.
